(19) United States
(12) Patent Application Publication
(10) Pub. No.: US 2025/0115922 A1
(43) Pub. Date: Apr. 10, 2025

(54) VIRULENCE PLASMIDS FOR AGROBACTERIUM MEDIATED EUKARYOTIC TRANSFORMATION

(71) Applicant: THE REGENTS OF THE UNIVERSITY OF CALIFORNIA, Oakland, CA (US)

(72) Inventors: Mitchell G. THOMPSON, Berkeley, CA (US); Allison N. PEARSON, Berkeley, CA (US); Patrick M. SHIH, Berkeley, CA (US)

(21) Appl. No.: 18/907,422

(22) Filed: Oct. 4, 2024

Related U.S. Application Data
(60) Provisional application No. 63/588,661, filed on Oct. 6, 2023.

(51) Int. Cl. C12N 15/82

(52) U.S. Cl. CPC C12N 15/8205

(57) ABSTRACT

The present invention provides for a nucleic acid encoding refactored minimized set of Agrobacterium virulence genes. The present invention provides for a method for introducing a nucleic acid of interest into a eukaryotic cell, the method comprises: (a) providing (i) a first nucleic acid encoding a refactored minimized set of Agrobacterium virulence genes operatively linked to one or more promoters; and (ii) a second nucleic acid comprising a nucleic acid of interest flanked by a left border and a right border; (b) introducing the first nucleic acid and the second nucleic acid into a target host cell; and, (c) the nucleic acid of interest is stably integrated into a genome of the target host cell.

### CROSS REFERENCE TO RELATED APPLICATIONS

This application claims the priority benefit of U.S. Provisional Application Nos. 63/588,661, filed Oct. 6, 2023, which is hereby incorporated by reference in its entirety.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of genetic transformation of eukayotic cells.

### BACKGROUND OF THE INVENTION

The genetic basis of pathogenesis can be challenging to study due to its highly polygenic nature as well as its dependence on both host and environmental factors 1. While advances in comparative and functional genomics have generated myriad hypotheses on how virulence and adaptations to specific hosts evolve 2,3, it is still challenging to isolate and validate specific genetic features that determine these traits 4. In an ideal system, researchers could evaluate the impact of specific regulatory or genetic changes; however, pleiotropic effects often complicate the conclusions drawn from traditional top-down approaches that rely solely on knockouts and complementation 5. As an alternative bottom-up approach, synthetic biology offers the ability to introduce synthetic regulatory control on a defined set of genetic elements. These strategies have been widely implemented in reconstituting relatively linear metabolic pathways 6,7, but apart from a few notable exceptions, they are rarely applied to more complex biological phenomena 8,9. A prerequisite for such “genetic refactoring” approaches includes identifying the genes necessary and sufficient for a given biological process 5, as well as having appropriate genetic tools in often non-model organisms 10. A problem unique to studying any host-pathogen interaction is that any synthetic regulatory elements utilized must also be robust in situ, i.e., in the context of infection, where very few genetic toolkits have been rigorously validated. Despite these challenges, work with both plant- and mammalian-associated bacteria has demonstrated that synthetic genetic constructs can be introduced to promote non-native interactions between host and microbe 11,12, indicating the feasibility of a complete synthetic refactoring of pathology. Nonetheless, genetically recapitulating complex biological phenomena within a host-associated environment has largely remained out of reach by synthetic biologists.

The plant pathogen Agrobacterium tumefaciens and other Rhizobium capable of causing either crown gall or hairy root disease have been extensively studied due to their unique pathology that has been leveraged for its novel biotechnology role in genetic transformations of eukaryotes 13. The hallmark of A. tumefaciens pathogenesis is the transfer of a protein-conjugated, single-stranded DNA molecule into the host genome. When genes from this “Transfer-DNA” (T-DNA) are expressed in the infected plant cell, the gene products produce phytohormones that result in the formation of a tumor in which the bacterium has privileged access to nutrients. The T-DNA and the majority of the virulence (vir) genes required to infect the plant are located on a single large tumor-inducing plasmid (pTi). A new era of plant genetics was ushered in when scientists domesticated this pathology by replacing the tumorigenic genes in the T-DNA with genes of interest. Today the T-DNA borders and genetic payloads to be delivered are most often housed on a smaller plasmid referred to as a binary vector enabling easy genetic manipulation of multitudes of plant and fungal species.

In parallel to elucidating the molecular factors involved in T-DNA transfer, researchers also recognized that different isolates of Agrobacterium have distinct host ranges, and these differences were largely determined by the pTi 14. By mining this natural diversity, strains with improved plant transformation properties for different plant species were quickly developed. More recently, groups have developed strains of Agrobacterium that contain additional vir genes originating from multiple pTi plasmids, harbored either on the binary vector (superbinary vectors) or on an additional stand-alone plasmid (ternary vectors), which have improved transformation of recalcitrant plants such as sorghum and maize 15-17. Precisely why some pTi are more efficient than others is largely unknown as they simultaneously differ in their vir gene composition and regulation, both of which can dramatically impact transformation between plants 18-20. A major source of variation between these plasmid families is the regulation of vir gene expression by the master regulatory two-component system VirA/G 20,21. VirA/G, in combination with other regulators, integrates multiple environmental signals to positively control the expression of all known pTi-located vir genes 22,23. On account of this pleiotropic regulatory schema, it is difficult to evaluate whether pathological phenotypes are a consequence of the presence of a specific vir gene or its strength of expression. Furthermore, little work has examined the impact of allelic diversity in most vir genes. Thus, to fully capture the impact of these many individual genetic variables involved in AMT a bottom-up synthetic genetic approach would be required to exert control not possible in natural systems.

### SUMMARY OF THE INVENTION

The present invention provides for a nucleic acid encoding refactored minimized set of Agrobacterium virulence genes. In some embodiments, the Agrobacterium virulence genes are operatively linked to one or more promoters.

The present invention provides for a method for introducing a nucleic acid of interest into a eukaryotic cell, the method comprises: (a) providing (i) a first nucleic acid encoding a refactored minimized set of Agrobacterium virulence genes operatively linked to one or more promoters; and (ii) a second nucleic acid comprising a nucleic acid of interest flanked by a left border and a right border; (b) introducing the first nucleic acid and the second nucleic acid into a target host cell; and, (c) the nucleic acid of interest is stably integrated into a genome of the target host cell.

The present invention provides for a method for constructing a refactored minimized set of Agrobacterium virulence genes, the method comprises ligating or synthesizing a nucleic acid encoding a refactored minimized set of Agrobacterium virulence genes. A refactored minimized set of Agrobacterium virulence genes has one or more virulence genes that are not essential for transfer of the nucleic acid of interest into the target host cell.

The present invention also provides for a vector comprising the nucleic acid of the present invention. In some embodiments, the vector is capable of stably integrating into a chromosome of a host cell or stably residing in a host cell. In some embodiments, the vector is an expression vector.

The present invention provides for a host cell comprising one or more vectors of the present invention.

In some embodiments, the first nucleic acid and the second nucleic acid reside on a single nucleic acid molecule, such as a vector, such a plasmid, capable of stably residing in a host cell. In some embodiments, the vector is a minimal refactored pTi plasmid. In some embodiments, the vector is capable of stably integrating into a chromosome of a host cell or stably residing in a host cell. In some embodiments, the vector is an expression vector.

In some embodiments, the refactored minimized set of Agrobacterium virulence genes comprises the following genes: (a) virB1, virB2, virB3, virB4, virB5, virB6, virB7, virB8, virB9, virB10, virB11, virD4, and virD12; and (b) (i) virE12; and/or (ii) virC12, virD5, and/or virE3.

In some embodiments, the refactored minimized set of Agrobacterium virulence genes comprises the following genes: (a) virB1, virB2, virB3, virB4, virB5, virB6, virB7, virB8, virB9, virB10, virB11, virD4, and virD12; (b) virE12; and (c) optionally virC12, virD5, and/or virE3.

In some embodiments, the refactored minimized set of Agrobacterium virulence genes comprises the following genes: (a) virB1, virB2, virB3, virB4, virB5, virB6, virB7, virB8, virB9, virB10, virB11, virD4, virD12, virE12, and virC12; and (b) optionally virD5, and/or virE3.

In some embodiments, the refactored minimized set of Agrobacterium virulence genes comprises the following genes: (a) virB1, virB2, virB3, virB4, virB5, virB6, virB7, virB8, virB9, virB10, virB11, virD4, virD12, virE12, virC12, virD5, and virE3.

In some embodiments, the refactored minimized set of Agrobacterium virulence genes comprises the genes described in Example 1 herein.

In some embodiments, the second nucleic acid is a vector, such a plasmid, capable of stably residing in a host cell. In some embodiments, the second nucleic acid further comprises: (1) a first selectable marker operatively linked to a eukaryotic promoter also flanked by the left border and the right border, (2) a second selectable marker operatively linked to a prokaryotic promoter, and/or (3) one or more origin of replication (ori), wherein each ori confers the capability of stable residence in a different host cell, for example, one ori confers stable residence in Escherichia coli, while another confers stable residence in an Agrobacterium.

In some embodiments, one or more of the vectors can stably reside in any bacteria, such a bacterium that is not A. tumefaciens/fabrum, for example, Escherichia coli or a Rhizobium cell, such as Rhizobium rhizogenes.

In some embodiments, the promoters are each independently constitutive or inducible. In some embodiments, the promoters are promoters described in Example 1 herein.

In some embodiments, the target host cell is a eukaryotic cell, such as a plant or fungal cell. In some embodiments, the plant is a tobacco plant. In some embodiments, the fungal cell is a Rhodosporidium cell, such as Rhodosporidium toruloides. In some embodiments, the fungal cell is torulosis's a yeast. In some embodiments, the yeast is Saccharomyces species, such as a Saccharomyces cerevisiae

The refactored minimized set of Agrobacterium virulence genes at least excludes: (1) the virA and virG genes, as these genes are regulatory genes; (2) the virB1 gene (Berger et al., J. Bacteriol. 176 (12): 3646-3660, 1994); and, (3) the virE12 gene (which can be replaced with the Agrobacterium rhizogenes GALLS gene (Hodges et al., J. Bacteriol. 191 (1): 355-364, 2009). In some embodiments, the refactored minimized set of Agrobacterium virulence genes excludes all of the vir genes that are not essential for virulence (such as vir genes that are not described in particular constructs described herein), and/or excludes elements of the native virulence plasmid that are not essential for virulence, such as its conjugal plasmid transfer system.

In some embodiments, the nucleic acid of interest encodes one or more genes of interest (GOI) each operatively linked to a promoter capable of expression in the target host cell.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1: Characterizing Synthetic Biology Toolkit in planta: A) Activity of constitutive promoters driving RFP from pGingerBK plasmid backbone. The x- and y-axes show RFP production from A. fabrum C58C1 3 days after infiltration in tobacco or Arabidopsis respectively (n=12). The color palette displays the activity of the same promoter in vitro (n=8). B) Transient expression of GFP from agroinfiltrated tobacco leaves (AU) log 2 transformed is shown on the y-axis. Different constitutive promoters used to complement a virE12 deletion mutant are shown as box and whisker plots with individual data points overlaid (n=64). Transformation by wild-type GV3101 and tobacco leaf without infiltration controls are shown. C) Proteomic spectral counts of VirE12 are shown when virE12 is expressed from different constitutive synthetic promoters (n=3) Rows D-G show characterization of P_{LacO}, P_{TetR}, P_{NahR}, P_{JungleExpress} respectively. From left to right Activity of inducible promoters driving RFP from pGingerBK plasmid backbone in tobacco (n=12), Arabidopsis (n=12), and in vitro (n=8). Inducer either not added (“None”), added at the half maximal induction concentration determined in vitro (“Mid”), or at the maximal induction concentration (“High”). The middle panel show the complementation of a virE12 deletion by different inducible promoters as measured by transient GFP expression shown on the y-axis after log 2 transformation (n=64). Inducer either not added (“None”), added at the half maximal induction concentration determined in vitro (“Mid”), or at the maximal induction concentration (“High”). The right panel shows proteomic spectral counts of VirE12 when expressed from different inducible promoters (n=3). Inducer either not added (“None”), added at the half maximal induction concentration determined in vitro (“Mid”), or at the maximal induction concentration (“High”).

FIG. 2: Quantitative assessment of vir gene impact on transformation: A) The virulence gene cluster from pTiC58. Known non-regulatory vir genes are shown in red, while regulatory vir genes are shown in blue. All other genes are grey. B) Effect of individual vir gene cluster deletion on tobacco transformation is measured by transient expression of GFP shown in log 2 transformed AU in pink, and complementation of the phenotype driven by P_{J23117} is shown in blue (n=64). Dashed pink line shows transformation by wild-type GV3101, while dashed blue line shows transformation by GV3101 expressing RFP from P_{J23117} as a control. C) Picture of a tobacco leaf expressing GFP delivered by a virD12 deletion mutant complemented from an IPTG inducible promoter with varying levels of induction indicated. Below shows transient GFP expressed in tobacco when transformed by virD12 deletion with different IPTG levels shown in log 2 transformed AU is shown on the y-axis, with the concentration of IPTG used to induce the promoter on the x-axis (n=64). Dashed line shows wild-type level transformation D) Complementation of vir gene deletion mutants that showed trends from an IPTG-inducible promoter using different strength constitutive promoters. Transient tobacco-expressed GFP shown in log 2 transformed AU is shown on the y-axis (n=64). Dashed line shows wild-type level transformation, colors of boxplots represent strength of constitutive promoters from FIG. 1 (Panel A).

FIG. 3: Impact of vir gene allele on tobacco transformation: B-K) Plots on the left show the effects of alleles of the indicated gene clusters in A. fabrum GV3101 deletion mutants complemented with pGinger based vectors. Box plots in pink show alleles that are statistically worse than the wild-type allele, box plots in blue show alleles that are statistically superior than the wild-type allele, and box plots in white show alleles that are statistically indistinguishable from the wild-type allele. Statistical significance was determined using a Bonferroni corrected T-test (p-value<0.05, n=64). Scatterplots on the right show the results of allele complementation assays in tobacco (y-axis) as a function of phylogenetic distance of each allele from the native allele (A. Fabrum C58). The p-values for both Pearson and Spearman correlation are shown above each plot.

FIG. 4: Synthetic refactoring of pTi: A) Genetic design of pDimples vectors. Variants of pDimples1 that only have either virC12 or virE12 (pDimples0.5), as well as variants of pDimples2 that have only virE3 or virD5 (pDimples1.5) were also constructed. B) Transient transformation of tobacco leaves by synthetic pTi plasmids harbored in A. fabrum C58C1 (n=64). The Y-axis shows log 2 transformed GFP (AU). C) Fluorescent microscopy of 6 mM tobacco leaf punches infiltrated with A. fabrum C58C1 harboring minimal refactored pTi plasmids as well as a binary vector for the expression of a nuclear localized mScarlet. D) Transformation of R. toruloides by synthetically refactored pTi plasmids harbored in A. fabrum C58C. The average number of transformants obtained in three transformations is shown by refactored strains, as well as a wild-type strain of A. fabrum GV3101 and A. fabrum C58C1 harboring a binary vector but no refactored pTi. E) Phylogenetic tree shows evolutionary history of Rhizobiales with the distance between A. fabrum C58 and R. rhizogenes D108/85 highlighted. Fluorescent microscopy of 6 mm tobacco leaf punches infiltrated with R. rhizogenes D108/85 harboring a binary vector for the expression of a nuclear localized mScarlet without (top) or with (bottom) pDimples1.0.

FIG. 5: Characterization of constitutive promoters: A) RFP expression from pGingerBK constitutive promoters within A. fabrum after infiltration into A. thaliana leaves 1 or 3 days post. Error bars show standard deviations (n=12). B) RFP expression from pGingerBK constitutive promoters within A. fabrum after infiltration into tobacco leaves 1 or 3 days post. Error bars show standard deviations (n=12). C) Scatterplot shows correlation between in vitro expression of constitutive promoters and expression within A. thaliana leaf tissue 3 days post infiltration. D) Scatterplot shows correlation between in vitro expression of constitutive promoters and expression within tobacco leaf tissue 3 days post infiltration E) Scatterplot shows correlation between expression of constitutive promoters within tobacco leaf tissue 3 days post infiltration and expression within A. thaliana leaf tissue 3 days post infiltration. F) Scatterplot shows correlation of RFP expression from pGingerBK constitutive promoters in E. coli and A. fabrum in vitro. Promoters used in this study are highlighted. Error bars show standard deviations (n=8).

FIG. 6: Dose response curves of the four inducible promoters used in this study. Graphs show RFP expression of A) Jungle Express B) NahR C) LacO and D) TetR (n=8). Dashed lines show fit to the Hill equation, and shaded area represents the confidence of the fit. E) Table shows relevant parameters of the Hill Equation fit.

FIG. 7: Graphs show expression of A. fabrum C58C1 RFP from inducible promoters within A. thaliana 1 (A) or 3 (B) days post infiltration, and tobacco 1 (C) or 3 (D) days post infiltration, at different levels of promoter induction (n=12).

FIG. 8: Impact of increasing vir gene expression on tobacco transient transformation. Graphs show complementation of vir gene cluster deletions of A. fabrum GV3101 with the IPTG inducible pGingerBS-LacO plasmid. The y-axis shows transient GFP production and the x-axis shows complementation at different levels of IPTG induction (n=64). Plots outlined in yellow show a positive correlation between induction and transformation, plots outlined in red show a negative correlation between induction and transformation, and plots outlined in gray show no correlation between induction and transformation. Dashed red lines on plots show a wild-type level of transformation.

FIG. 9: Growth rate of A. fabrum GV3101 vir gene cluster deletion strains complemented with IPTG inducible pGingerBS-LacO plasmid. Graph shows the maximal logarithmic phase growth rate of strains grown in a microplate reader at different levels of IPTG induction (n=4). GV3101 wild-type expressing RFP is shown as a control. Error bars represent standard deviations.

FIG. 10: Complementing virB1-5 and virB6-11 deletion mutants: A) Effect of knocking out either virB1-5 or virB6-11 is measured by transient expression of GFP shown in log 2 transformed AU (n=64). B) Graphs show complementation of either virB1-5 or virB6-11 gene cluster deletions of A. fabrum GV3101 with the IPTG inducible pGingerBS-LacO plasmid. The y-axis shows transient GFP production and the x-axis shows complementation at different levels of IPTG induction (n=64). Dashed red lines on plots show a wild-type level of transformation. C) Growth of wild-type A. fabrum GV3101 expressing RFP from IPTG inducible pGingerBS-LacO plasmid (black), a virB1-5 deletion mutant expressing virB1-5 from an IPTG inducible pGingerBS-LacO plasmid (red), and a virB6-11 deletion mutant expressing virB6-11 from an IPTG inducible pGingerBS-LacO plasmid (red), with either 0 (left panel) or 75 uM (right panel) IPTG added (n=4). D) Evaluating constitutive promoter complementation of either virB1-5 or virB6-11 deletion mutants of A. fabrum GV3101. The y-axis shows log 2 transformed GFP (AU) from transient tobacco infiltrations, x-axis represents different constitutive promoters tested (n=64). Dashed red lines on plots show a wild-type level of transformation.

FIG. 11: Optimization of virB1-11 complementation: A) Evaluating different complementation vectors of a virB1-11 deletion mutant of A. fabrum GV3101. The y-axis shows log 2 transformed GFP (AU) from transient tobacco infiltrations, x-axis represents different constitutive promoters tested (n=64). Genetic diagrams to the right are color coordinated with the boxplots to the left. B) Comparing P_{virB}, P_{LacO}, and P_{J23101} complementation vectors of a virB1-11 deletion mutant of A. fabrum GV3101. The y-axis shows log 2 transformed GFP (AU) from transient tobacco infiltrations, x-axis represents promoters tested (n=64).

FIG. 12: Impact of vir gene allele on tobacco transformation: B-K) Plots on the left show the effects of alleles of the indicated gene clusters in A. fabrum GV3101 deletion mutants complemented with pGinger based vectors that resulted in improved transformation. Box plots in pink show alleles that are statistically worse than the wild-type allele, box plots in blue show alleles that are statistically superior than the wild-type allele, and box plots in white show alleles that are statistically indistinguishable from the wild-type allele. Statistical significance was determined using a Bonferroni corrected T-test (p-value<0.05, n=64). Scatterplots on the right show the results of allele complementation assays in tobacco (y-axis) as a function of phylogenetic distance of each allele from the native allele (A. Fabrum C58). The p-values for both Pearson and Spearman correlation are shown above each plot.

FIG. 13: Selective pressure on vir genes. For each conserved vir gene the diversifying (positive selection shown in red) and negative (purifying selection shown in blue) selection are shown for each residue of the protein using dN/dS analysis. Resides that have no significant selection are shown in black.

FIG. 14: Evaluation of combinatorial vir allele complementation: A) Genetic Diagrams of the pLoki1.0 and pLoki2.0 vectors. B) Table shows allelic composition of pLoki1 vectors. C) Table shows allelic composition of pLoki2 vectors. D) Graph shows complementation of a virC2-virE3 gene cluster deletion of A. fabrum GV3101 with the pLoki plasmids. The y-axis shows transient GFP production and the x-axis shows complementation with different variants (n=64). E) Graph shows complementation of a virC2-virE3 gene cluster deletion of A. fabrum GV3101 with the pLoki plasmids harboring only wild-type (C58) alleles. The y-axis shows transient GFP production and the x-axis shows complementation with different variants (n=64). F) Composite complementation virC2-virE3 gene cluster deletion with pLoki vectors that have virD5 pTiBo542 alleles versus those harboring the wild-type virD5 allele. G) Composite complementation virC2-virE3 gene cluster deletion with pLoki vectors that have virD4 pTiBo542 alleles versus those harboring the wild-type virD4 allele. H) Composite complementation virC2-virE3 gene cluster deletion with pLoki vectors that have virE3 pTiT60_94 alleles versus those harboring the wild-type virE3 allele.

FIG. 15: Evaluation of refactored pTi plasmids. A) Transient transformation of tobacco leaves as measured by GFP production by synthetic pTi plasmids harbored either in A. fabrum C58C1 or GV3101 ΔvirA-E3 (n=64). The Y-axis shows log 2 transformed GFP (AU). B) Graph shows the impact of different amounts of IPTG on the ability of pDimples1.0 and pDimples 2.0 to enable tobacco transient transformation of A. fabrum C58C1 (n=64) C) Graph shows the impact of increasing the expression of virD4 by replacing the PJ23114 promoter with PJ23117 in pDimples 1.0 harbored by C58C1. The y-axis shows tobacco transient GFP production (n=64) D) Graph shows the number of colonies obtained by transforming R. toruloides with A. fabrum C58C1 pDimples1.0 or pDimples 2.0 with and without the addition of IPTG to induction media (n=3) E) PCR confirmation of 15 randomly selected colonies of R. toruloides transformed by A. fabrum C58C1 pDimples 2.0 with primers specific to inserted T-DNA. The first non-ladder well shows product amplified directly from binary vector plasmid, and the last non-ladder well shows negative result amplified from wild-type R. toruloides. F) Graph shows the amount of fluorescence from tobacco leaf punches infiltrated with either a naturally cured strain of R. rhizogenes or the same strain harboring pDimples1.0 (n=12).

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A “constitutive promoter” is one that is capable of initiating transcription in nearly all cell types, whereas a “cell type-specific promoter” initiates transcription only in one or a few particular cell types or groups of cells forming a tissue. In some embodiments, the promoter is secondary cell wall-specific and/or fiber cell-specific. A “fiber cell-specific promoter” refers to a promoter that initiates substantially higher levels of transcription in fiber cells as compared to other non-fiber cells of the plant. A “secondary cell wall-specific promoter” refers to a promoter that initiates substantially higher levels of transcription in cell types that have secondary cell walls, e.g., lignified tissues such as vessels and fibers, which may be found in wood and bark cells of a tree, as well as other parts of plants such as the leaf stalk. In some embodiments, a promoter is fiber cell-specific or secondary cell wall-specific if the transcription levels initiated by the promoter in fiber cells or secondary cell walls, respectively, are at least 3-fold, 4-fold, 5-fold, 6-fold, 7-fold, 8-fold, 9-fold, 10-fold, 50-fold, 100-fold, 500-fold, 000-fold higher or more as compared to the transcription levels initiated by the promoter in other tissues, resulting in the encoded protein substantially localized in plant cells that possess fiber cells or secondary cell wall, e.g., the stem of a plant. Non-limiting examples of fiber cell and/or secondary cell wall specific promoters include the promoters directing expression of the genes IRX1, IRX3, IRX5, IRX7, IRX8, IRX9, IRX10, IRX14, NST1, NST2, NST3, MYB46, MYB58, MYB63, MYB83, MYB85, MYB103, PAL1, PAL2, C3H, CcOAMT, CCR1, F5H, LAC4, LAC17, CADc, and CADd. See, e.g., Turner et al 1997; Meyer et al 1998; Jones et al 2001; Franke et al 2002; Ha et al 2002; Rohde et al 2004; Chen et al 2005; Stobout et al 2005; Brown et al 2005; Mitsuda et al 2005; Zhong et al 2006; Mitsuda et al 2007; Zhong et al 2007a, 2007b; Zhou et al 2009; Brown et al 2009; McCarthy et al 2009; Ko et al 2009; Wu et al 2010; Berthet et al 2011. In some embodiments, a promoter is substantially identical to a promoter from the lignin biosynthesis pathway. A promoter originated from one plant species may be used to direct gene expression in another plant species.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “host cell” of “host organism” is used herein to refer to a living biological cell that can be transformed via insertion of an expression vector.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

Described herein is the refactoring of multiple virulence (vir) plasmids of Agrobacterium sp. so that one can specifically titrate the expression strength and allele variants of a minimized vir gene complement. Agrobacterium tumefaciens and related bacterium are invaluable tools for the transformation of a myriad of eukaryotes. However, the transformation efficiency is highly dependent on the strain of Agrobacterium, as well as the host. Optimization of transformation often involves the time consuming process of evaluating various naturally derived strains of Agrobacterium in many conditions that induce their natural virulence complement that is required for genetic transformation.

These required vir genes in all laboratory strains of Agrobacterium is controlled using natural inducers, transduced by the master regulatory system VirA/G. Because all the genes are controlled by this single regulator, it is difficult to specifically increase or decrease expression of vir genes that may impact transformation efficiency across the many eukaryotic organisms that are engineered via Agrobacterium. Furthermore, the large plasmids that house the majority of the vir genes impose a significant metabolic burden to the host bacterium.

The present invention reduces the large virulence plasmids down to a much smaller subset of genes required for transformation, that have been divorced from native induction and have instead been placed under the control of inducible promoters that allow a greater dynamic range of expression. These systems can readily interchange vir alleles from phylogentically distinct Agrobacterium isolates, and evaluate their impact on transformational efficiency in a dose-dependent manner. These minimized plasmids can be expressed from a variety of gram-negative bacterial hosts, and can optimize and improve transformation outcomes in eukaryotic hosts.

In all of the strains of Agrobacterium used for transformation to date, a critical step of the transformation protocol is the expression of the virulence (vir) genes. Together these genes catalyze the formation of the T-DNA complex which is then shuttled into the host nucleus and either transiently expressed, or stably integrated into the chromosome. To date natural isolates that vary the expression strength and allelic composition of their vir complement vary greatly in their transformational efficiency of different hosts. It is well known that certain strains of Agrobacterium are required for optimal transformation of specific eukaryotes, such as how the common laboratory strain EHA105 generally outperforms other strains in the transformation of fungi.

A major limitation of the reliance on natural isolate derivatives is that induction of all known vir genes is controlled by a single master regulator. Different strains have evolved different expression patterns of their vir genes, presumably as an adaptation to different host niches. It is unlikely, however, that evolution has produced strains that are optimally suited for laboratory transformation. Previous work has addressed this by adding additional copies of virulence genes and regulators that increase vir gene expression which can increase transformational efficiency in some hosts. However, these innovations still require activation through natural transduction mechanisms that sense environmental factors such as pH, glucose, and phenolic compounds, such as acetosyringone. This regulatory schema lends the bacteria susceptible to expression interference from hosts, an inability to tune expression so limiting vir genes are overexpressed, and an expression of vir genes that are not necessary and therefore only impose a metabolic burden on the bacteria.

The present invention avoids all of these pitfalls of relying on natural induction through the minimization and refactoring of the vir genes. To accomplish this, the normally about 200 kb virulence plasmid (pTi) is divided into two independently replicating plasmids that harbor a reduced complement of vir genes (various combinations of virB1-5, virB6-11, virC12, virD12, virD4, virE12, virD5, virE3, and virF). These clusters are expressed independently of one another via orthogonal inducible systems each with a greater range of expression than the existing native regulation. Any complement of known virulence alleles can be used, allow for the creation of hybrid vir gene complements that may prove more effective in the transformation of particular crops. The use of broad host range origins on both the refactored virulence plasmids will permit vir gene expression from a phylogentically diverse range of gram-negative bacteria which can affect the plant's ability to mount an innate immune response. In conjunction with standard vectors, these refactored vir plasmids enable rapid host-specific optimization of either transient or stable transformation of eukaryotic hosts.

Major obstacles to the implementation of this technology have been the identification of both specific alleles and expression levels that optimize transformation outcomes in specific hosts. Similarly, the optimization of vir gene expression to optimize either for transient expression or for “high-quality” single insertional events is non-trivial and non-obvious.

Herein is described the successful engineering of components required to refactor minimized virulence plasmids comprised of alleles from highly diverged Agrobacterium strains. Herein is demonstrated the expression of key virulence protein(s) at ranges above and below that of native regulation, and that this can be used to improve upon the transformation efficiency of common laboratory strains in tobacco.

The most important use of the present invention is enabling the transformation of traditionally recalcitrant fungi, plants, and other eukaryotic organisms. Enabling genetic transformation of such organisms will allow for entities to introduce modifications into the genomes of organisms that possess superior natural traits innately which could have wide ranging impacts on agriculture, renewable chemical production, and medicine. The present invention enables more rapid optimization of transformation outcomes in species that are already somewhat tractable.

The present invention has the following advantages: the refactored plasmids of the present invention are completely divorced from the native VirA/G regulatory system and work as a standalone virulence system that does not require the presence of any disarmed pTi or pRi virulence plasmids. In all previous work, induction of the vir genes generally requires the native VirA/G regulation and the presence of a disarmed virulence plasmid.

Another key difference between the present invention and previous systems is that the present invention can selectively express particular vir gene alleles at specific expression levels in different bacterial host contexts. In systems that rely on VirA/G systems to induce native plasmids, there is no ability to selectively turn on particular vir genes or vary the allelic composition as the plasmids are naturally derived.

The present invention provides for plasmids that are dramatically smaller than the natural pTi. This lowers the metabolic burden on the bacterial cell that allow for greater vir gene expression if required. The present invention provides for further minimization that eliminates elements of the native virulence plasmid such as its conjugal plasmid transfer system that will enhance the biocontainment properties of strains used in the present invention.

In some embodiments, when the target host cell is a plant cell, the promoter is a tissue-specific promoter. Examples of tissue-specific promoters under developmental control include promoters that initiate transcription only (or primarily only) in certain tissues, such as vegetative tissues, cell walls, including e.g., roots or leaves. A variety of promoters specifically active in vegetative tissues, such as leaves, stems, roots and tubers are known. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarn promoter of the gene encoding a globulin from a major taro (Colocasia esculenta L. Schott) corm protein family, tarin (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier, FEBS Lett. 415:91-95, 1997). A ribulose bisphosphate carboxylase promoters expressed almost exclusively in mesophyll cells in leaf blades and leaf sheaths at high levels (e.g., Matsuoka, Plant J. 6:311-319, 1994), can be used. Another leaf-specific promoter is the light harvesting chlorophyll a/b binding protein gene promoter (see, e.g., Shiina, Plant Physiol. 115:477-483, 1997; Casal, Plant Physiol. 116:1533-1538, 1998). The Arabidopsis thaliana myb-related gene promoter (Atmyb5) (Li, et al., FEBS Lett. 379:117-121 1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize (e.g., Busk et al., Plant J. 11:1285-1295, 1997) can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are kn1-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

In some embodiments, the promoter is substantially identical to the native promoter of a promoter that drives expression of a gene involved in secondary wall deposition. Examples of such promoters are promoters from IRX1, IRX3, IRX5, IRX8, IRX9, IRX14, IRX7, IRX10, GAUT13, or GAUT14 genes. Specific expression in fiber cells can be accomplished by using a promoter such as the NST1 promoter and specific expression in vessels can be accomplished by using a promoter such as VND6 or VND7. (See, e.g., PCT/US2012/023182 for illustrative promoter sequences). In some embodiments, the promoter is a secondary cell wall-specific promoter or a fiber cell-specific promoter. In some embodiments, the promoter is from a gene that is co-expressed in the lignin biosynthesis pathway (phenylpropanoid pathway). In some embodiments, the promoter is a C4H, C3H, HCT, CCR1, CAD4, CAD5, F5H, PAL1, PAL2, 4CL1, or CCoAMT promoter. In some embodiments, the tissue-specific secondary wall promoter is an IRX1, IRX3, IRX5, IRX8, IRX9, IRX14, IRX7, IRX10, GAUT13, GAUT14, or CESA4 promoter. Suitable tissue-specific secondary wall promoters, and other transcription factors, promoters, regulatory systems, and the like, suitable for this present invention are taught in U.S. Patent Application Pub. Nos. 2014/0298539, 2015/0051376, and 2016/0017355.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In some embodiments, each GOI is operatively linked to a promoter that is activated by the transcription activator. In some embodiments, each GOI is a biosynthetic gene that expresses an enzyme that catalyzes the biosynthesis of a compound of interest, or an intermediate thereof.

### REFERENCES CITED

1. Scholthof, K.-B. G. (2007). The disease triangle: pathogens, the environment and society. Nat. Rev. Microbiol.5, 152-156. 10.1038/nrmicro1596.
2. Laabei, M., and Massey, R. (2016). Using functional genomics to decipher the complexity of microbial pathogenicity. Curr. Genet.62, 523-525. 10.1007/s00294-016-0576-4.
3. Merhej, V., Georgiades, K., and Raoult, D. (2013). Postgenomic analysis of bacterial pathogens repertoire reveals genome reduction rather than virulence factors. Brief. Funct. Genomics12, 291-304. 10.1093/bfgp/elt015.
4. Pfeilmeier, S., Caly, D. L., and Malone, J. G. (2016). Bacterial pathogenesis of plants: future challenges from a microbial perspective: Challenges in Bacterial Molecular Plant Pathology. Mol. Plant Pathol.17, 1298-1313. 10.1111/mpp.12427.
5. Deutscher, D., Meilijson, I., Schuster, S., and Ruppin, E. (2008). Can single knockouts accurately single out gene functions? BMC Syst. Biol.2, 50. 10.1186/1752-0509-2-50.
6. Calgaro-Kozina, A., Vuu, K. M., Keasling, J. D., Loqué, D., Sattely, E. S., and Shih, P. M. (2020). Engineering plant synthetic pathways for the biosynthesis of novel antifungals. ACS Cent. Sci.6, 1394-1400. 10.1021/acscentsci.0c00241.
7. Ro, D.-K., Paradise, E. M., Ouellet, M., Fisher, K. J., Newman, K. L., Ndungu, J. M., Ho, K. A., Eachus, R. A., Ham, T. S., Kirby, J., et al. (2006). Production of the antimalarial drug precursor artemisinic acid in engineered yeast. Nature440, 940-943. 10.1038/nature04640.
8. Budin, I., and Keasling, J. D. (2019). Synthetic biology for fundamental biochemical discovery. Biochemistry58, 1464-1469. 10.1021/acs.biochem.8b00915.
9. Temme, K., Zhao, D., and Voigt, C. A. (2012). Refactoring the nitrogen fixation gene cluster from Klebsiella oxytoca. Proc Natl Acad Sci USA 109, 7085-7090. 10.1073/pnas.1120788109.
10. Kim, N. M., Sinnott, R. W., and Sandoval, N. R. (2020). Transcription factor-based biosensors and inducible systems in non-model bacteria: current progress and future directions. Curr. Opin. Biotechnol.64, 39-46. 10.1016/j.copbio.2019.09.009.
11. Sorg, R. A., Gallay, C., Van Maele, L., Sirard, J.-C., and Veening, J.-W. (2020). Synthetic gene-regulatory networks in the opportunistic human pathogen Streptococcus pneumoniae. Proc Natl Acad Sci USA 117, 27608-27619. 10.1073/pnas.1920015117.
12. Geddes, B. A., Kearsley, J. V. S., Huang, J., Zamani, M., Muhammed, Z., Sather, L., Panchal, A. K., diCenzo, G. C., and Finan, T. M. (2021). Minimal gene set from Sinorhizobium (Ensifer) meliloti pSymA required for efficient symbiosis with Medicago. Proc Natl Acad Sci USA 118. 10.1073/pnas.2018015118.
13. Nester, E. W. (2014). Agrobacterium: nature's genetic engineer. Front. Plant Sci.5, 730. 10.3389/fpls.2014.00730.
14. Thomashow, M. F., Panagopoulos, C. G., Gordon, M. P., and Nester, E. W. (1980). Host range of Agrobacterium tumefaciens is determined by the Ti plasmid. Nature283, 794-796. 10.1038/283794a0.
15. Anand, A., Bass, S. H., Wu, E., Wang, N., McBride, K. E., Annaluru, N., Miller, M., Hua, M., and Jones, T. J. (2018). An improved ternary vector system for Agrobacterium-mediated rapid maize transformation. Plant Mol. Biol.97, 187-200. 10.1007/s11103-018-0732-y.
16. Anand, A., Che, P., Wu, E., and Jones, T. J. (2019). Novel ternary vectors for efficient sorghum transformation. Methods Mol. Biol.1931, 185-196. 10.1007/978-1-4939-9039-9_13.
17. Komari, T., Takakura, Y., Ueki, J., Kato, N., Ishida, Y., and Hiei, Y. (2006). Binary vectors and super-binary vectors. Methods Mol. Biol.343, 15-41. 10.1385/1-59745-130-4:15.
18. Jarchow, E., Grimsley, N. H., and Hohn, B. (1991). virF, the host-range-determining virulence gene of Agrobacterium tumefaciens, affects T-DNA transfer to Zea mays. Proc Natl Acad Sci USA88, 10426-10430. 10.1073/pnas.88.23.10426.
19. Hansen, G., Das, A., and Chilton, M. D. (1994). Constitutive expression of the virulence genes improves the efficiency of plant transformation by Agrobacterium. Proc Natl Acad Sci USA 91, 7603-7607. 10.1073/pnas.91.16.7603.
20. Turk, S. C., Nester, E. W., and Hooykaas, P. J. (1993). The virA promoter is a host-range determinant in Agrobacterium tumefaciens. Mol. Microbiol.7, 719-724. 10.1111/j.1365-2958.1993.tb01162.x.
21. Raineri, D. M., Boulton, M. I., Davies, J. W., and Nester, E. W. (1993). VirA, the plant-signal receptor, is responsible for the Ti plasmid-specific transfer of DNA to maize by Agrobacterium. Proc Natl Acad Sci USA 90, 3549-3553. 10.1073/pnas.90.8.3549.
22. Krishnamohan, A., Balaji, V., and Veluthambi, K. (2001). Efficient vir gene induction in Agrobacterium tumefaciens requires virA, virG, and vir box from the same Ti plasmid. J. Bacteriol.183, 4079-4089. 10.1128/JB.183.13.4079-4089.2001.
23. Lin, Y.-H., Gao, R., Binns, A. N., and Lynn, D. G. (2008). Capturing the VirA/VirG TCS of Agrobacterium tumefaciens. Adv. Exp. Med. Biol.631, 161-177. 10.1007/978-O-387-78885-2_11.
24. Schuster, L. A., and Reisch, C. R. (2021). A plasmid toolbox for controlled gene expression across the Proteobacteria. Nucleic Acids Res.49, 7189-7202. 10.1093/nar/gkab496.
25. Qian, Y., Kong, W., and Lu, T. (2021). Precise and reliable control of gene expression in Agrobacterium tumefaciens. Biotechnol. Bioeng.118, 3962-3972. 10.1002/bit.27872.
26. Pearson, A. N., Thompson, M. G., Kirkpatrick, L. D., Ho, C., Vuu, K. M., Waldburger, L. M., Keasling, J. D., and Shih, P. M. (2023). The pGinger Family of Expression Plasmids. Microbiol. Spectr.11, e0037323. 10.1128/spectrum.00373-23.
27. Zhou, A., Kirkpatrick, L. D., Ornelas, I. J., Washington, L. J., Hummel, N. F. C., Gee, C. W., Tang, S. N., Barnum, C. R., Scheller, H. V., and Shih, P. M. (2023). A suite of constitutive promoters for tuning gene expression in plants. ACS Synth. Biol. 12, 1533-1545. 10.1021/acssynbio.3c00075.
28. Zipfel, C., Kunze, G., Chinchilla, D., Caniard, A., Jones, J. D. G., Boller, T., and Felix, G. (2006). Perception of the bacterial PAMP EF-Tu by the receptor EFR restrictsAgrobacterium-mediated transformation. Cell125, 749-760. 10.1016/j.cell.2006.03.037.
29. Zhang, X., Hooykaas, M. J. G., van Heusden, G. P., and Hooykaas, P. J. J. (2022). The translocated virulence protein VirD5 causes DNA damage and mutation during Agrobacterium-mediated transformation of yeast. Sci. Adv.8, eadd3912. 10.1126/sciadv.add3912.
30. Zhang, X., van Heusden, G. P. H., and Hooykaas, P. J. J. (2017). Virulence protein VirD5 of Agrobacterium tumefaciens binds to kinetochores in host cells via an interaction with Spt4. Proc Natl Acad Sci USA 114, 10238-10243. 10.1073/pnas.1706166114.
31.Wang, K., Herrera-Estrella, A., and Van Montagu, M. (1990). Overexpression of virD1 and virD2 genes in Agrobacterium tumefaciens enhances T-complex formation and plant transformation. J. Bacteriol.172, 4432-4440. 10.1128/jb.172.8.4432-4440.1990.
32. Song, M., Sukovich, D. J., Ciccarelli, L., Mayr, J., Fernandez-Rodriguez, J., Mirsky, E. A., Tucker, A. C., Gordon, D. B., Marlovits, T. C., and Voigt, C. A. (2017). Control of type III protein secretion using a minimal genetic system. Nat. Commun.8, 14737. 10.1038/ncomms14737.
33. Weisberg, A. J., Davis, E. W., Tabima, J., Belcher, M. S., Miller, M., Kuo, C.-H., Loper, J. E., Grünwald, N. J., Putnam, M. L., and Chang, J. H. (2020). Unexpected conservation and global transmission of agrobacterial virulence plasmids. Science368. 10.1126/science.aba5256.
34.Idnurm, A., Bailey, A. M., Cairns, T. C., Elliott, C. E., Foster, G. D., Ianiri, G., and Jeon, J. (2017). A silver bullet in a golden age of functional genomics: the impact of Agrobacterium-mediated transformation of fungi. Fungal Biol. Biotechnol.4, 6. 10.1186/s40694-017-0035-0.
35. Hooykaas, P. J. J., van Heusden, G. P. H., Niu, X., Reza Roushan, M., Soltani, J., Zhang, X., and van der Zaal, B. J. (2018). Agrobacterium-Mediated Transformation of Yeast and Fungi. Curr. Top. Microbiol. Immunol.418, 349-374. 10.1007/82_2018_90.
36. Lang, J., Gonzalez-Mula, A., Taconnat, L., Clement, G., and Faure, D. (2016). The plant GABA signaling downregulates horizontal transfer of the Agrobacterium tumefaciens virulence plasmid. New Phytol.210, 974-983. 10.1111/nph.13813.
37. Liu, P., and Nester, E. W. (2006). Indoleacetic acid, a product of transferred DNA, inhibits vir gene expression and growth of Agrobacterium tumefaciens C58. Proc Natl Acad Sci USA 103, 4658-4662. 10.1073/pnas.0600366103.
38. Michielse, C. B., Hooykaas, P. J. J., van den Hondel, C. A. M. J. J., and Ram, A. F. J. (2005). Agrobacterium-mediated transformation as a tool for functional genomics in fungi. Curr. Genet.48, 1-17. 10.1007/s00294-005-0578-0.
39. Yang, F., Li, G., Felix, G., Albert, M., and Guo, M. (2023). Engineered Agrobacterium improves transformation by mitigating plant immunity detection. New Phytol.237, 2493-2504. 10.1111/nph.18694.
40. Raman, V., Rojas, C. M., Vasudevan, B., Dunning, K., Kolape, J., Oh, S., Yun, J., Yang, L., Li, G., Pant, B. D., et al. (2022). Agrobacterium expressing a type III secretion system delivers Pseudomonas effectors into plant cells to enhance transformation. Nat. Commun. 13, 2581. 10.1038/s41467-022-30180-3.
41. Ham, T. S., Dmytriv, Z., Plahar, H., Chen, J., Hillson, N. J., and Keasling, J. D. (2012). Design, implementation and practice of JBEI-ICE: an open source biological part registry platform and tools. Nucleic Acids Res.40, e141. 10.1093/nar/gks531.
42. Chen, J., Densmore, D., Ham, T. S., Keasling, J. D., and Hillson, N. J. (2012). DeviceEditor visual biological CAD canvas. J. Biol. Eng.6, 1. 10.1186/1754-1611-6-1.
43. Hillson, N. J., Rosengarten, R. D., and Keasling, J. D. (2012). j5 DNA assembly design automation software. ACS Synth. Biol.1, 14-21. 10.1021/sb2000116.
44. Gibson, D. G., Young, L., Chuang, R.-Y., Venter, J. C., Hutchison, C. A., and Smith, H. O. (2009). Enzymatic assembly of DNA molecules up to several hundred kilobases. Nat. Methods 6, 343-345. 10.1038/nmeth.1318.
45. Engler, C., Kandzia, R., and Marillonnet, S. (2008). A one pot, one step, precision cloning method with high throughput capability. PLOS ONE 3, e3647. 10.1371/journal.pone.0003647.
46. Green, M. R., and Sambrook, J. (2012). Molecular Cloning: A Laboratory Manual (Fourth Edition), Volume 1, 2 & 3 4th ed. (Cold Spring Harbor Laboratory Press).
47. Kámán-Tóth, E., Pogány, M., Dankó, T., Szatmári, Á., and Bozsó, Z. (2018). A simplified and efficient Agrobacterium tumefaciens electroporation method. 3 Biotech8, 148. 10.1007/s13205-018-1171-9.
48. Thompson, M. G., Blake-Hedges, J. M., Cruz-Morales, P., Barajas, J. F., Curran, S. C., Eiben, C. B., Harris, N. C., Benites, V. T., Gin, J. W., Sharpless, W. A., et al. (2019). Massively Parallel Fitness Profiling Reveals Multiple Novel Enzymes in Pseudomonas putida Lysine Metabolism. MBio10. 10.1128/mBio.02577-18.
49. Shanks, R. M. Q., Kadouri, D. E., MacEachran, D. P., and O'Toole, G. A. (2009). New yeast recombineering tools for bacteria. Plasmid62, 88-97. 10.1016/j.plasmid.2009.05.002.
50. Geiselman, G. M., Kirby, J., Landera, A., Otoupal, P., Papa, G., Barcelos, C., Sundstrom, E. R., Das, L., Magurudeniya, H. D., Wehrs, M., et al. (2020). Conversion of poplar biomass into high-energy density tricyclic sesquiterpene jet fuel blendstocks. Microb. Cell Fact.19, 208. 10.1186/s12934-020-01456-4.
51. Zhang, S., Skerker, J. M., Rutter, C. D., Maurer, M. J., Arkin, A. P., and Rao, C. V. (2016). Engineering Rhodosporidium toruloides for increased lipid production. Biotechnol. Bioeng.113, 1056-1066. 10.1002/bit.25864.
52. Gin, J., Chen, Y., and J Petzold, C. (2020). Chloroform-Methanol Protein Extraction for Gram-negative Bacteria (High Throughput) v1. 10.17504/protocols.io.bfx6jpre.
53. Chen, Y., Gin, J., and J Petzold, C. (2021). Discovery proteomic (DDA) LC-MS/MS data acquisition and analysis v2. 10.17504/protocols.io.buthnwj6.
54. Weisberg, A. J., Wu, Y., Chang, J. H., Lai, E.-M., and Kuo, C.-H. (2023). Virulence and ecology of agrobacteria in the context of evolutionary genomics. Annu. Rev. Phytopathol.61, 1-23. 10.1146/annurev-phyto-021622-125009.
55.Ranwez, V., Douzery, E. J. P., Cambon, C., Chantret, N., and Delsuc, F. (2018). MACSE v2: toolkit for the alignment of coding sequences accounting for frameshifts and stop codons. Mol. Biol. Evol.35, 2582-2584. 10.1093/molbev/msy 159.
56. Pond, S. L. K., Frost, S. D. W., and Muse, S. V. (2005). HyPhy: hypothesis testing using phylogenies. Bioinformatics21, 676-679. 10.1093/bioinformatics/bti079.
57. Nguyen, L.-T., Schmidt, H. A., von Haeseler, A., and Minh, B. Q. (2015). IQ-TREE: a fast and effective stochastic algorithm for estimating maximum-likelihood phylogenies. Mol. Biol. Evol.32, 268-274. 10.1093/molbev/msu300.
58. mwaskom/seaborn: v0.9.0 (July 2018)|Zenodo webpage for: doi.org/10.5281/zenodo.1313201.
59. Matplotlib: A 2D Graphics Environment-IEEE Journals & Magazine webpage for: doi.org/10.1109/MCSE.2007.55.
60. Jones, E., Oliphant, T., Peterson, P., and Others SciPy: Open source scientific tools for Python.
61. Gramfort, A., Luessi, M., Larson, E., Engemann, D. A., Strohmeier, D., Brodbeck, C., Parkkonen, L., and Hämäläinen, M. S. (2014). MNE software for processing MEG and EEG data. Neuroimage86, 446-460. 10.1016/j.neuroimage.2013.10.027.
62. Katoh, K., and Standley, D. M. (2013). MAFFT multiple sequence alignment software version 7: improvements in performance and usability. Mol. Biol. Evol.30, 772-780. 10.1093/molbev/mst010.
63. Price, M. N., Dehal, P. S., and Arkin, A. P. (2010). FastTree 2-approximately maximum-likelihood trees for large alignments. PLOS ONE 5, e9490. 10.1371/journal.pone.0009490.
64. Sukumaran, J., and Holder, M. T. (2010). DendroPy: a Python library for phylogenetic computing. Bioinformatics26, 1569-1571. 10.1093/bioinformatics/btq228.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Genetically Refactored Agrobacterium-Mediated Transformation

Agrobacterium, a plant pathogen that can naturally transform plant cells, is the most important tool currently used to genetically modify plants. While many genetic and biochemical factors involved in this complex process are known, the specific mechanisms that govern transformation efficiency and plant host range are poorly defined. Central to this is that genetic diversity between the tumor-inducing plasmids (pTi), which encode most of the machinery required for transformation, convolutes genotype-to-phenotype correlations. Here we bypass these difficulties by generating minimized, genetically refactored pTi that impose synthetic regulation over a defined set of genes. Based on a comprehensive characterization of genetic variables that govern transformation, we designed synthetic pTi constructs capable of both plants and fungal transformation. We further demonstrate that our synthetic pTi can be functionally ported into distantly related Rhizobium and enable plant transformation. Our reductionist approach demonstrates how bottom-up engineering can be used to dissect and elucidate the genetic basis of complex biological traits, and may lead to the development of strains of bacteria more capable of transforming recalcitrant plant species of societal importance.

Despite the multitude of technical challenges associated with engineering synthetically encoded AMT, the potential of a deeper understanding of the process of plant transformation warrants such efforts. Here we overcome these challenges by validating a set of genetic tools that allow for reliable control of bacterial gene expression within the plant environment, exhaustively and quantitatively characterize the genetic contributions to AMT using traditional top-down genetics, and then synthesizing this data to generate synthetic vectors, divorced from native regulation, capable of plant transformation. This represents a critical first step in better understanding AMT as we lay the framework for understanding highly specific genotype-to-phenotype connections in a complex host-microbe interaction.

### Results and Discussion

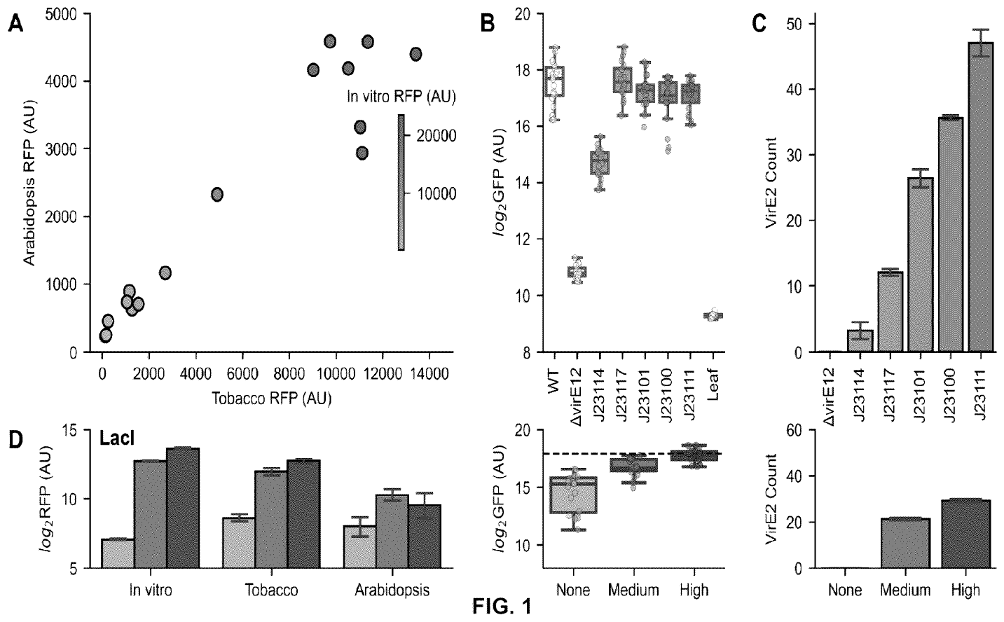

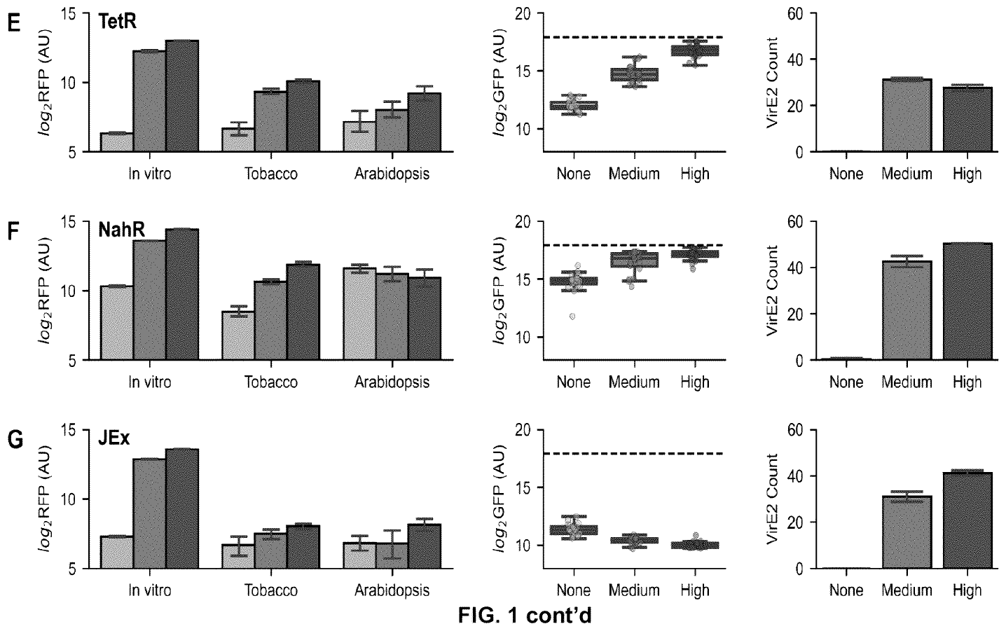

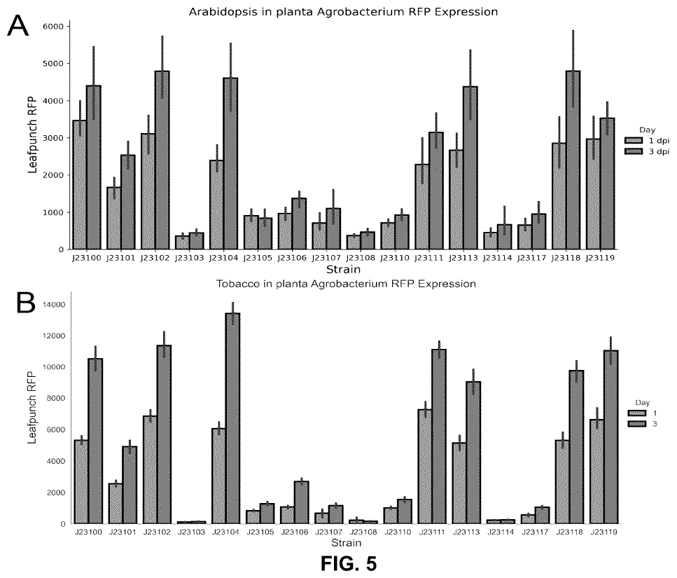

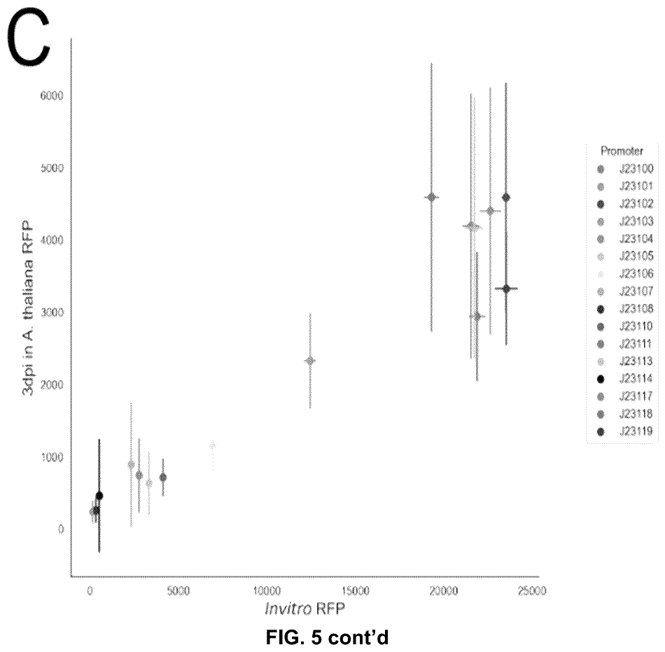

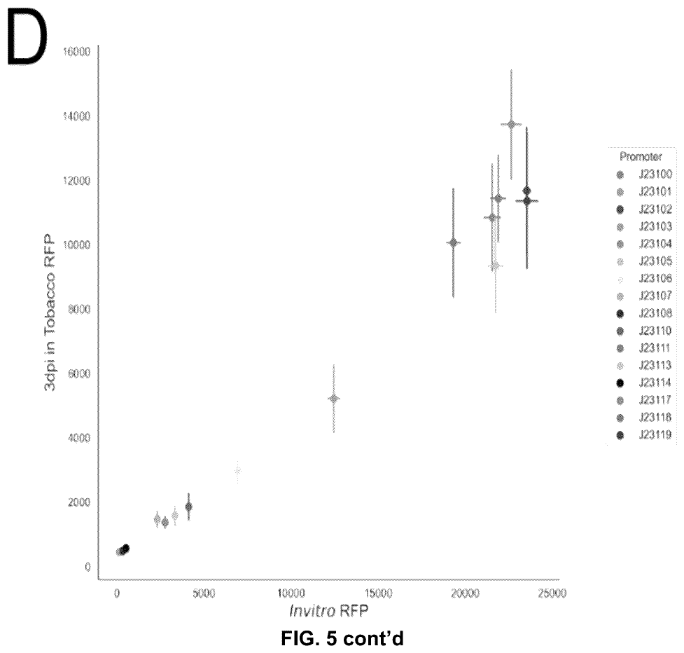

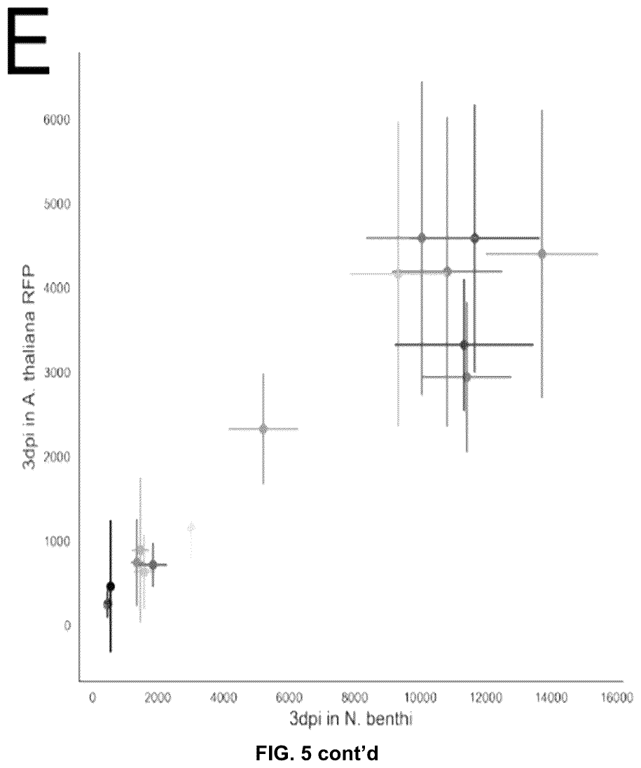

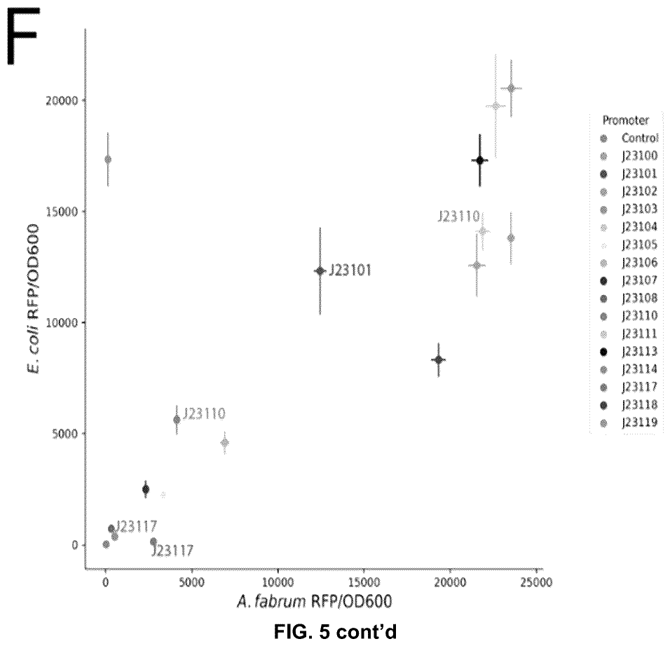

Developing a genetic toolkit to control bacterial gene expression in planta. A recurring challenge in synthetic biology has been translating genetic circuits developed in vitro into more heterogeneous environments in situ. Whether this be the result of scaling up a microbial factor from a test-tube to a fermentation tank, or deploying living medicine within a patient, environmental change can have dramatic impact on genetically engineered organisms. Many in vitro synthetic biology designs take advantage of small-molecule inducible promoters, which offers a range of expression options from a single design, compared to static expression levels from single constitutive promoter. However, dynamic environments such as plant tissue may interfere with inducible promoter systems by making signaling molecules biologically unavailable through degradation or sequestration, thus dramatically limiting their potential usefulness. Recent work by multiple groups characterized inducible promoters in Agrobacterium, though none were evaluated while the bacteria was in planta, and further there was no systematic characterization of constitutive promoters 24,25. To better understand how to control bacterial gene expression within plants we used the pGinger suite of plasmids we evaluated the activity of 16 synthetic constitutive, and 4 inducible promoters in rich media 26, as well as in the leaf tissue of Nicotiana benthamiana and Arabidopsis thaliana. Bacterial constitutive promoter activity correlated highly between leaf tissues from both plants, which both correlated to observed in vitro activity (FIG. 1, Panel A, FIG. 5). We then choose five promoters with a range of expression strengths (P_{J23114}, P_{J23117}, P_{J23101}, P_{J23100}, and P_{J23111}) to complement a virE12 deletion mutation in the common Agrobacterium fabrum laboratory strain GV3101, which is derived from A. fabrum C58. Using transiently expressed GFP from a medium strength plant promoter in N. benthamiana as a measure of transformation 27, we observed that all promoters stronger than the weakest, P_{J23114}, were able to complement leaf transformation back to wild-type levels (FIG. 1, Panel B). Proteomics analysis of the ΔvirE12 complementation strains confirmed that expression of VirE12 correlated with predictions based on RFP expression (FIG. 1, Panel C), revealing that relatively weak constitutive promoters may be sufficient to reconstitute vir gene expression from pBBR1 origin plasmids.

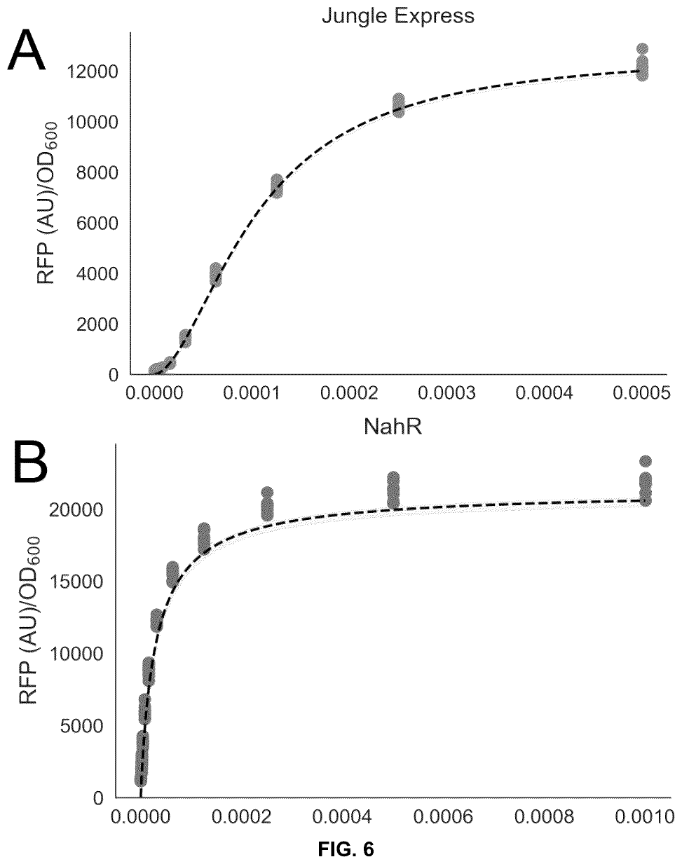

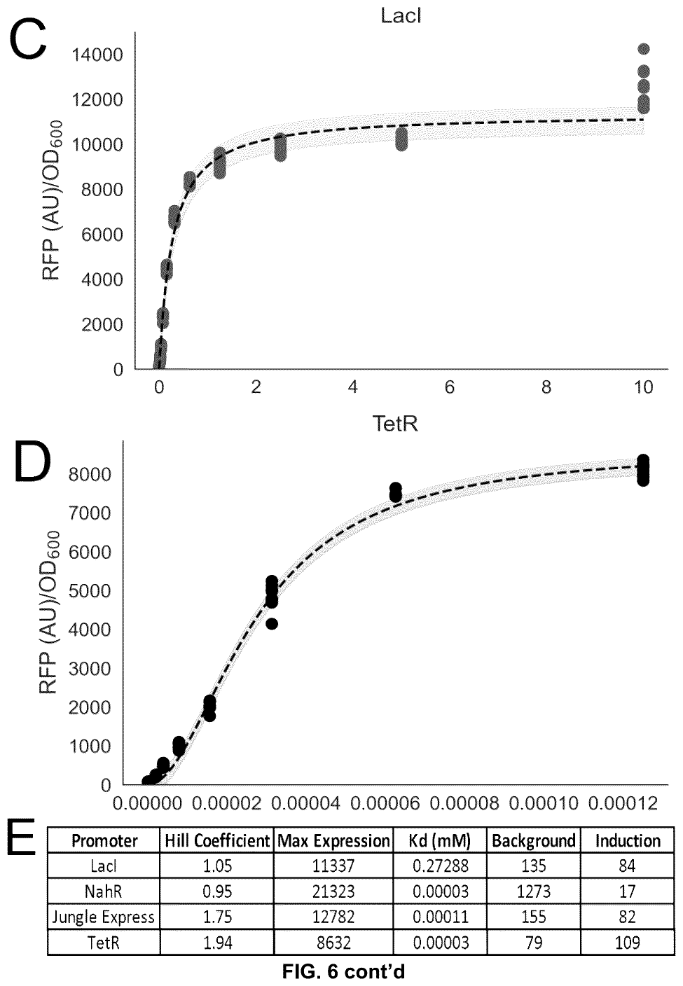

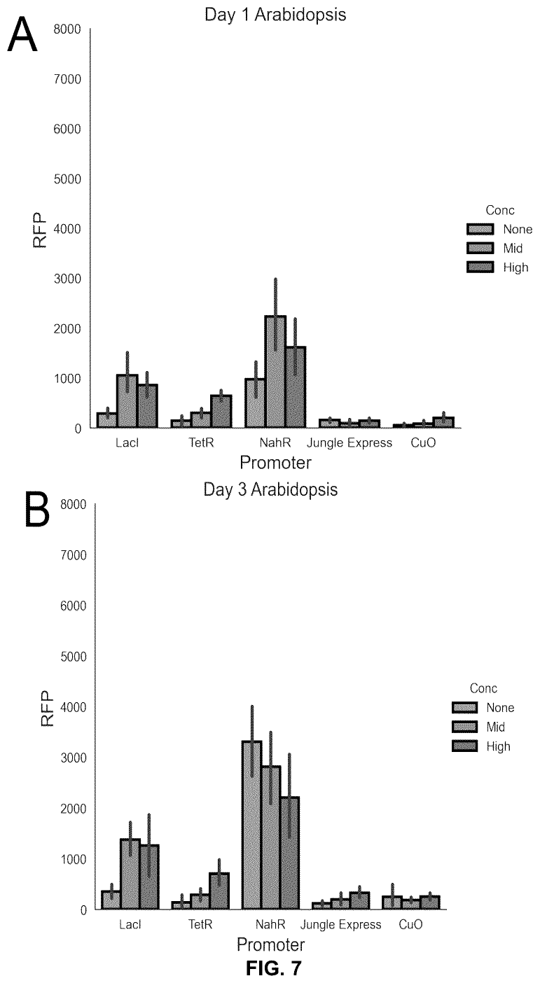

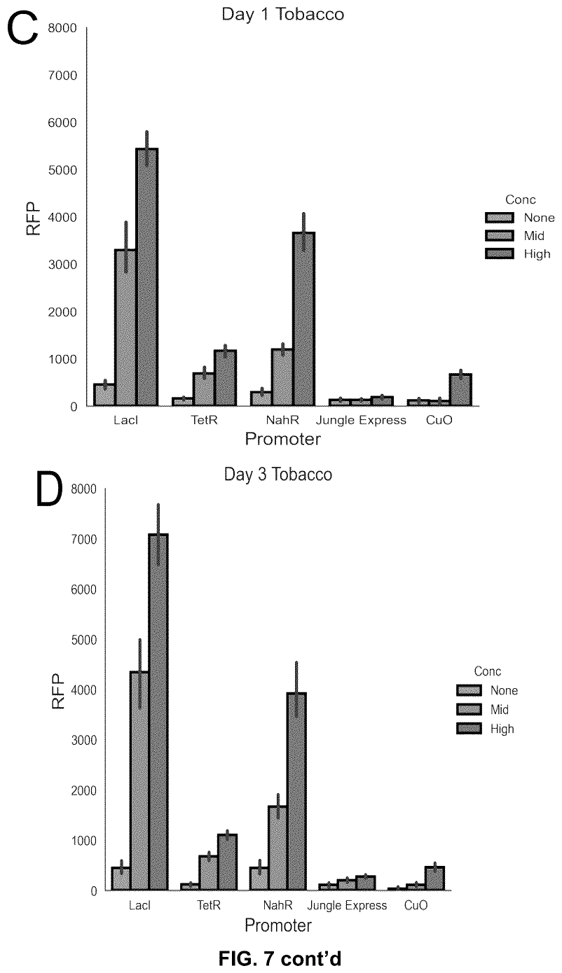

As inducible promoters would allow for dynamic control of gene expression strength, and thus limit the number of potential genetic designs needed to evaluate the impact of gene expression on AMT, we then evaluated the expression of RFP from four inducible promoter systems from the pGinger suite (P_{LacO}, P_{TetR}, P_{Jungle Express}, and P_{NahR}) in culture media as well as N. benthamiana and A. thaliana leaves, where the inducing compound was mixed with a bacterial suspension before infiltration into leaf tissue. While each of these systems displayed inducible expression in culture media (FIG. 6), only P_{LacO} and P_{TetR} showed consistent inducibility in both plant species (FIG. 1 (Panels D-G), FIG. 7). Conversely, the P_{Jungle Express} promoter showed poor induction in both plant species, and P_{NahR} was expressed even in the absence of an added inducer within A. thaliana leaf tissue. These results demonstrate the importance of validating each promoter in its intended environment. Though functional in vitro the crystal violet inducer of P_{Jungle Express} may be rapidly bound to plant tissue and thus not biologically available. Conversely the salicylic acid inducer of P_{NahR} is generated by plants that can mount an immune response to A. fabrum such as A. thaliana, and thus may not be ideal for exerting orthogonal control of gene expression within different plants 28. After testing all four promoter systems to complement a ΔvirE12 mutation, only the LacI inducible promoter with the highest amount of added ligand tested was able to recover transformation back to wild type levels (FIG. 1, Panel D). While proteomics from cultures indicated that the levels of VirE2 expressed from inducible promoters were similar to the constitutive promoters, the plant-specific utility of individual promoters suggests they may be less useful for designing general functioning genetic circuits across plant environments. As P_{LacO} showed the best plant orthogonality and ability to complement a virE12 mutation, further designs requiring inducibility utilized the IPTG inducible promoter.

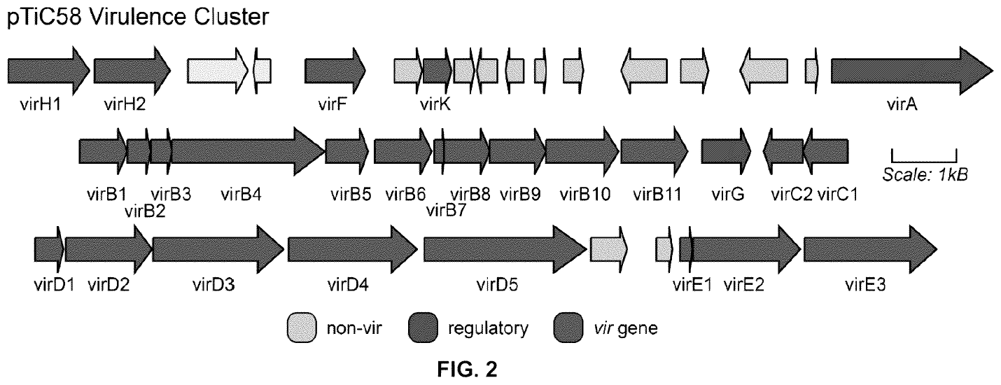

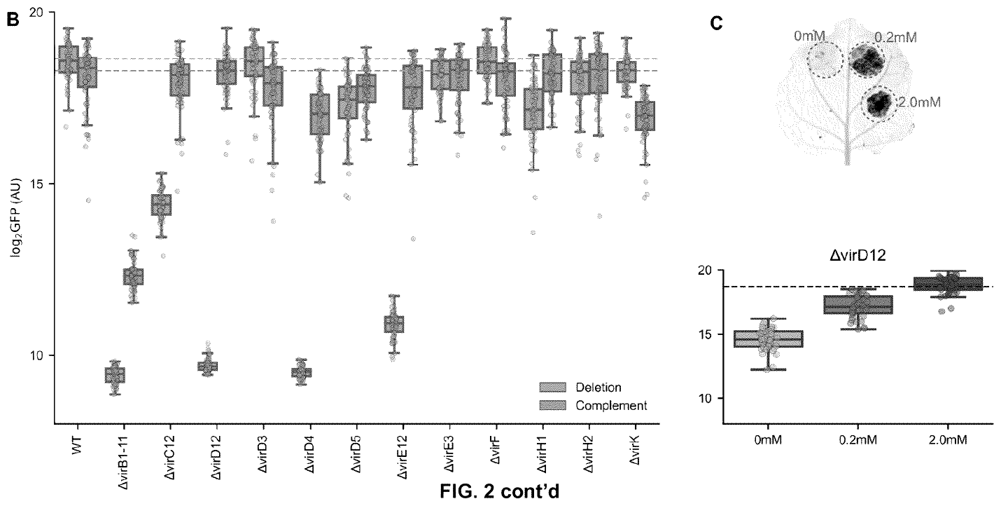

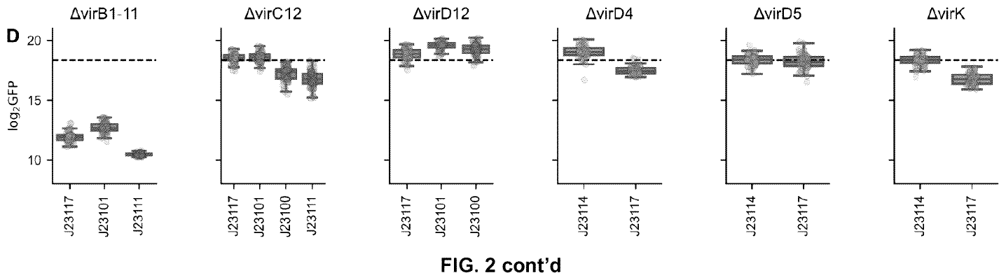

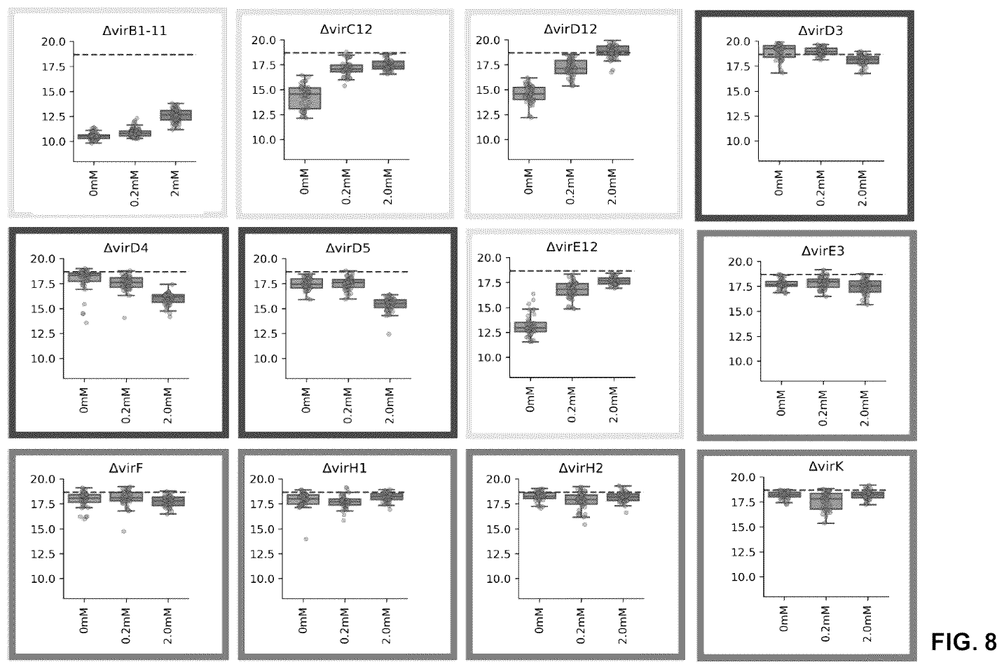

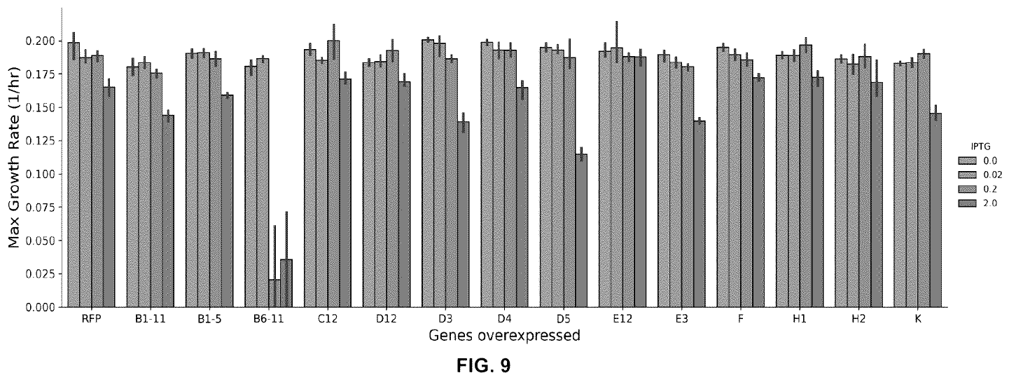

A quantitative understanding of the genetic contributions to AMT. To systematically assess the contributions individual vir genes have on plant transformation, we developed a quantitative virulence assay to measure the efficiency of T-DNA transfer into plant cells. To accomplish this, we first generated internal, in-frame deletion mutants of known functional non-regulatory vir gene clusters in A. fabrum GV3101: virB1-11, virC12, virD12, virD3, virD4, virD5, virE12, virE3, virF, virH1, virH2, and virK (FIG. 2, Panel A). Using our transient GFP expression assay in N. benthamiana leaves, we observed that deletion of virB1-11, virC12, virD12, virD4, or virE12 resulted in over 90% reductions in transformation efficiency; furthermore, loss of virD5, virE3, virF, virH1, virH2, or virK statistically reduced transformation efficiency compared to wild-type, while deletion of virD3 or virF showed no significant reduction in transformation efficiency (FIG. 2, Panel B). Plasmid complementation of these deletions using relatively weak promoter P_{J23117} restored wild-type transformation efficiencies in all deletion strains except virB1-11, virD4, virD5, and virK (FIG. 2, Panel B). To explore the effect of different expression levels on transformation efficiency, we then complemented each mutation with the inducible P_{LacO} promoter. Three phenotypes were observed; the virB1-11, virC12, virD12, and virE12 complementation strains had increasing transformation efficiency with increased induction; the virD3, virD4, and virD5 complementation strains had decreasing transformation efficiency as induction increased; the virE3, virF, virH1, virH2, and virk complementation strains showed no response to increasing induction (FIG. 2, Panel C, FIG. 8). Some of the decrease in transformation observed as virD5 expression increases may be due to toxicity to the bacterium, though similar significant toxicity was not observed with increased expression of virD3 or virD4 (FIG. 9). Previous work has shown that overexpression of virD5 resulted in acute toxicity in eukaryotes, where it is localized to the nucleus 29,30, and it is unclear if it is exerting its toxicity via a similar mechanism within the bacterial cell.

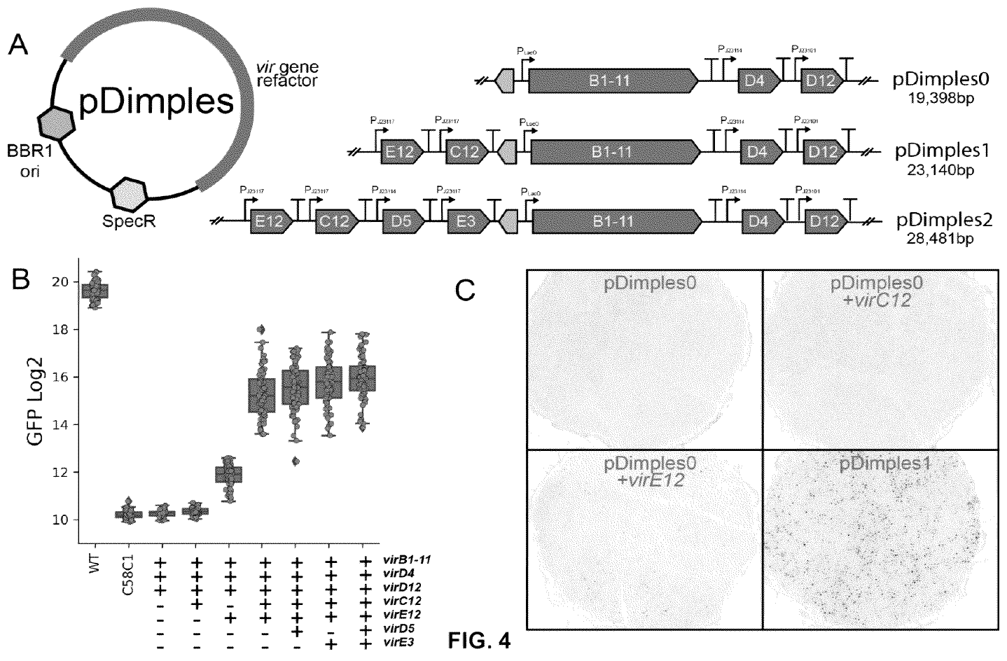

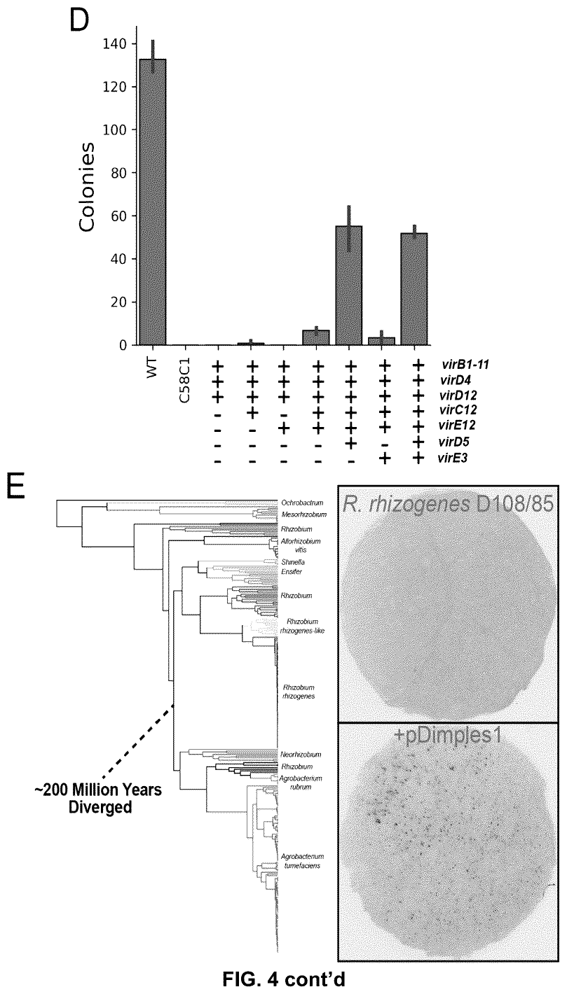

Based on these results, we used constitutive promoters stronger or weaker than second weakest P_{J23117} to optimize the expression of each vir gene cassette (FIG. 4, Panel D). Strong expression of virD12 improved transformation compared to wild-type by 135%. These results are in line with previous reports that overexpression of virD12 improves transformation 31. Conversely, lower expression of virD4 improved transformation 72% over wild-type. There was no statistical improvement of transformation by increasing the expression of virC12, though expression from the stronger P_{J23100} and P_{J23101} promoters decreased transformation. Expression of virD5 and virK from the weak P_{J23114} promoter was able to restore wild-type level transformation efficiency. Overall, these results demonstrate that transformation efficiency is highly sensitive to the expression strength of nearly all vir genes we evaluated, necessitating precise tuning for optimal DNA transfer. While expressing virB1-11 from the strong P_{J23101} promoter improved transformation over complementation using P_{J23117}, complementation from the strongest promoter tested, P_{J23111}, resulted in a significant reduction in transformation, suggesting that high-level expression may be toxic to the bacterium. Additionally, unlike other gene clusters, which were all complemented back to at least wild-type levels of transformation, we were only able to achieve ˜2% of wild-type transformation in a ΔvirB1-11 genetic background. The virB operon encodes the type 4 secretion system (T4SS), and previous studies genetically reconstructing secretion systems demonstrated the difficulty associated with engineering efficient transport 32, suggesting that a great deal of optimization may be required to efficiently express the virB1-11 type 4 secretion system (T4SS) of A. fabrum.

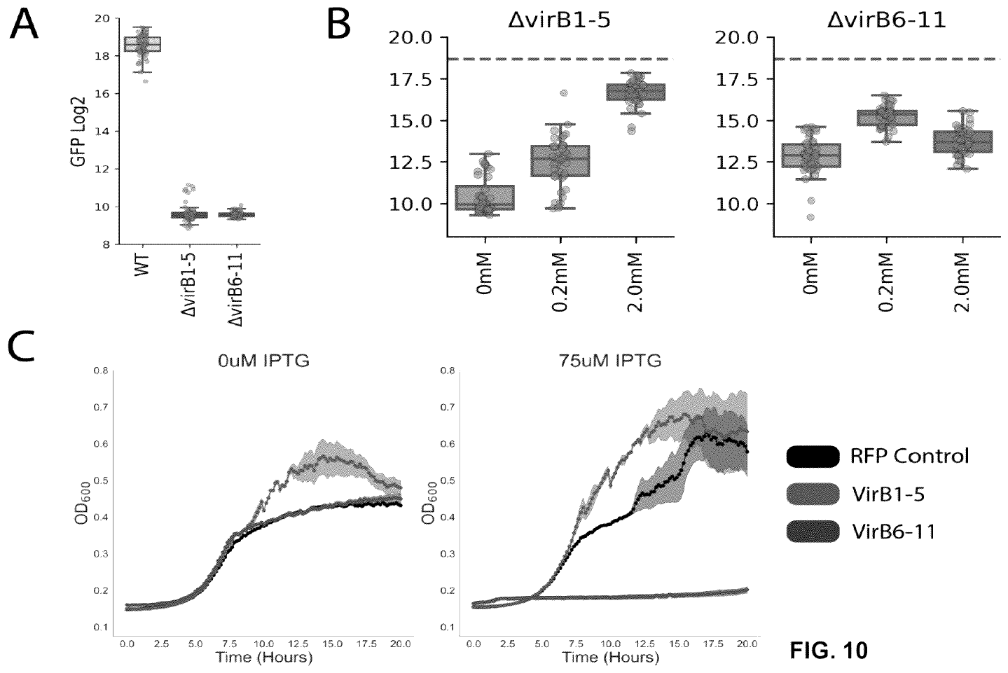

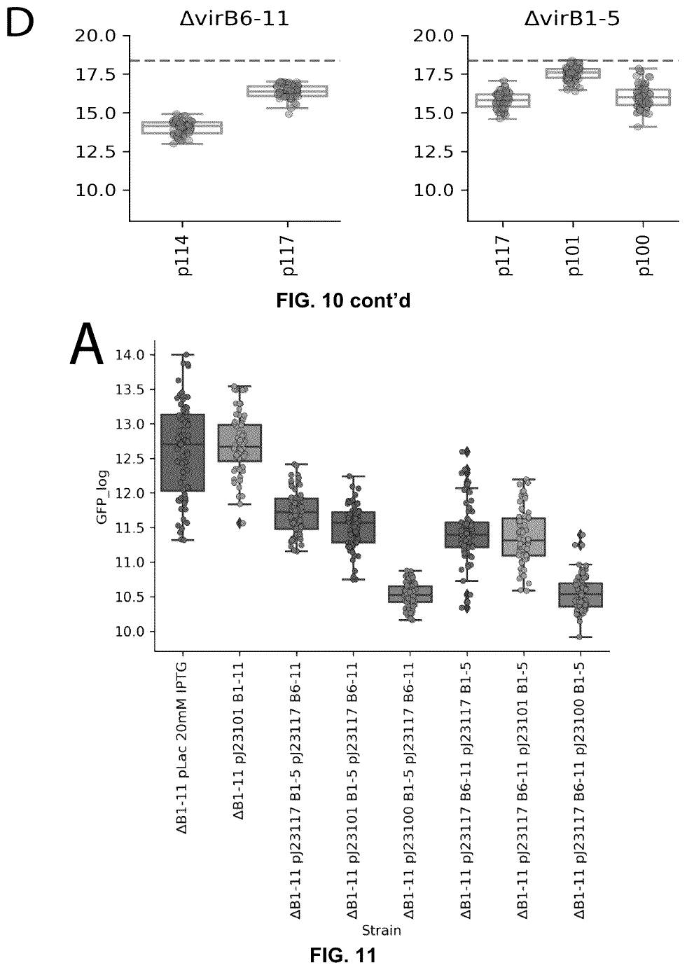

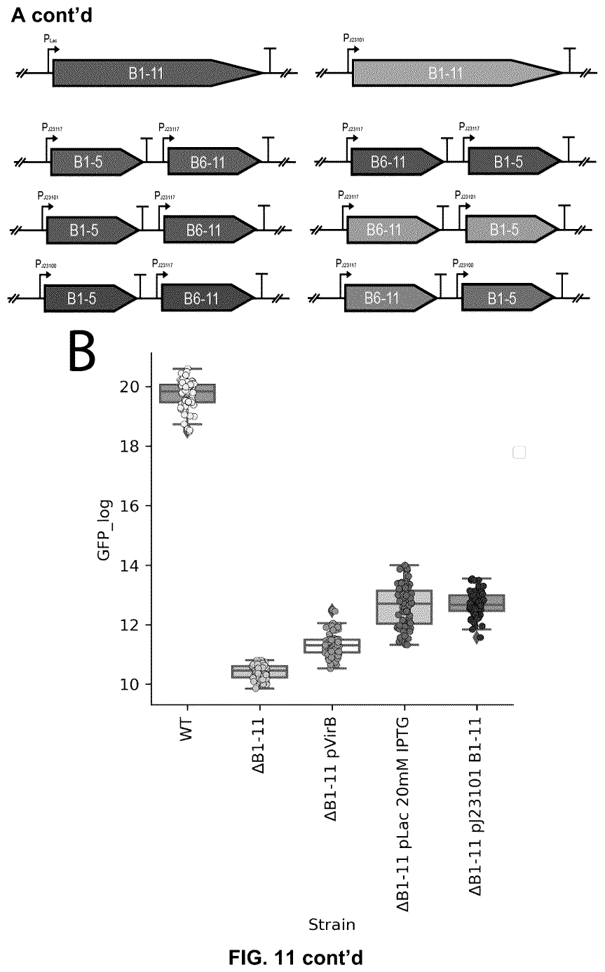

In an attempt to improve virB complementation, we explored whether breaking the cluster into segments would improve our ability to complement the virB cluster. We knocked out virB1-5 and virB6-11 individually and attempted to complement these smaller mutations. Both of the smaller mutations predictably abolished transformation (FIG. 10, Panel A). Using the P_{LacO} inducible promoter, virB1-5 showed a linear improvement of transformation as more IPTG was added; however, virB6-11 complementation plateaued at the median inducer concentration tested, with the highest level of induction causing a sharp decrease in transformation (FIG. 10, Panel B). The decrease in transformation is likely due to the extreme toxicity associated with virB6-11 being expressed without the other T4SS genes, which greatly compromised growth in a plate reader assay (FIG. 10, Panel C). Constitutive complementation assays revealed that optimal promoters to complement these deletions were the middle strength P_{J23101} for virB1-5 which yielded ˜60% of wild-type transformation, and the relatively weak P_{J23117} for virB6-11 which yielded ˜25% complementation (FIG. 10, Panel D). Based on this data we designed synthetic virB1-11 complementation vectors that drove virB1-5 of of three different promoters (weak-P_{J23117}, medium-P_{J23101}, and strong-P_{J23100}), and virB6-11 from P_{J23117} with this cassette both downstream and upstream of virB1-5 (FIG. 11, Panel A). However, none of these vectors could complement as well as when virB1-11 was expressed in its entirety, with the vectors driving virB1-5 from the strong P_{J23100} performing particularly poorly (FIG. 11, Panel A). To assess the performance of our synthetic complementation of virB1-11 against the native P_{virB}, we cloned the entire virB1-11 operon in addition to its intergenic upstream and downstream DNA into a promoterless pGinger backbone. While this vector was able to complement transformation above the ΔvirB1-11 parent, it was still significantly less than both virB1-11 expressed from P_{LacO}, as well as virB1-11 driven from P_{J23101} (FIG. 11, Panel B). These results suggest that the differing ratios between vir gene expression from the remaining pTi and virB1-11 expression from the pGinger plasmid may compromise our ability to complement the deletion of the T4SS in this specific genetic system, and require most vir genes to be expressed from the same vector.

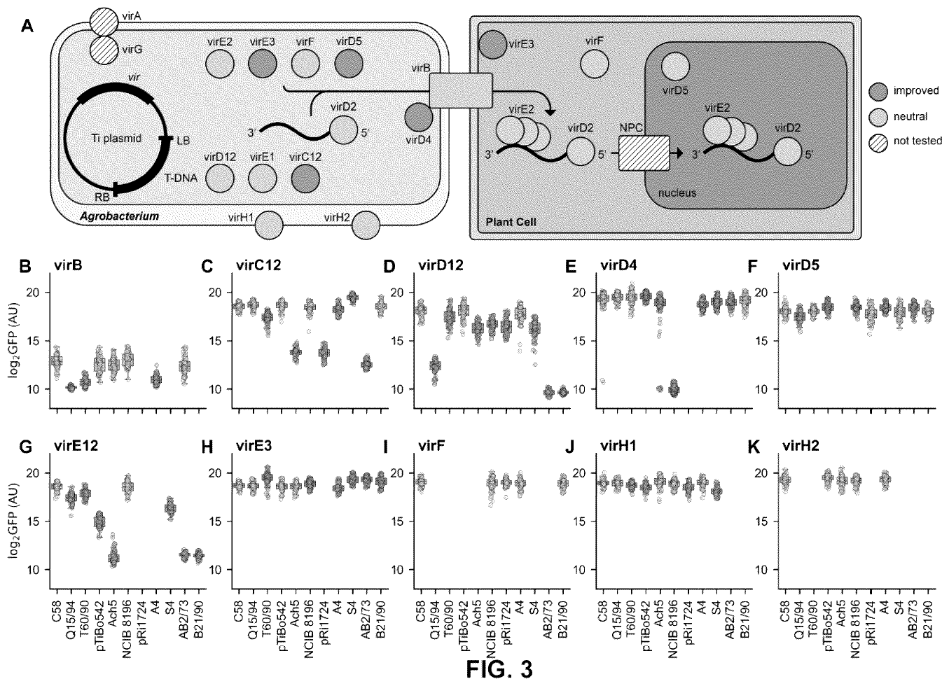

Impact of vir gene allelic variation on AMT. In many synthetically engineered metabolic pathways, multiple homologs of an enzyme are often evaluated for superior flux towards the final product. To our knowledge there has never been a systematic effort to determine whether specific homologs of a non-regulatory vir gene are able to improve transformation efficiency. In fact only recently would such an undertaking be feasible as the evolutionary history of the pTi/pRi plasmids was resolved in 2020, showing 9 distinct lineages of plasmids existing 33. Based on these phylogenies we sought to identify alleles of vir genes that could improve transformation, as well as determine whether phylogenetic distance between homologs plays a role in the ability of vir genes to function together. As AMT relies on multiple interactions between vir genes we reasoned that co-evolution may limit the ability of distantly related homologs from functioning with one another. (FIG. 3, Panel A). To measure the effect allelic variation plays in plant transformation, we synthesized phylogenetically diverse alleles from each of the 9 pTi/pRi (Table S1; which is found in the webpage for: biorxiv.org/content/10.1101/2023.10.13.561914v1.supplementary-material) families and evaluated their ability to complement deletion mutations of virB1-11, virC12, virD12, virD4, virD5, virE12, virE3, virH1, virH2, and virF in a tobacco transient expression system (FIG. 3, Panels B-K). Of these clusters, we identified alleles of virC12 (91% improvement), virD4 (13% improvement), virD5 (35% improvement), and virE3 (76% improvement) that resulted in improved complementation compared to the wild-type allele (C58). For virD5 4/9 alleles tested improved upon the wild type (FIG. 3, Panel F) and for virE3 4/9 tested also improved transformation compared to the native strain (FIG. 3, Panel H). However, for the critical vir genes virC12 (FIG. 3, Panel C), virD12 (FIG. 3, Panel D), and virE12 (FIG. 3, Panel G) the majority of homologs tested significantly reduced transformation. These results suggest that while homologs exist that improve transformation, there are likely design rules imposed by evolution that prevent random combinatorial assembly of vir genes.

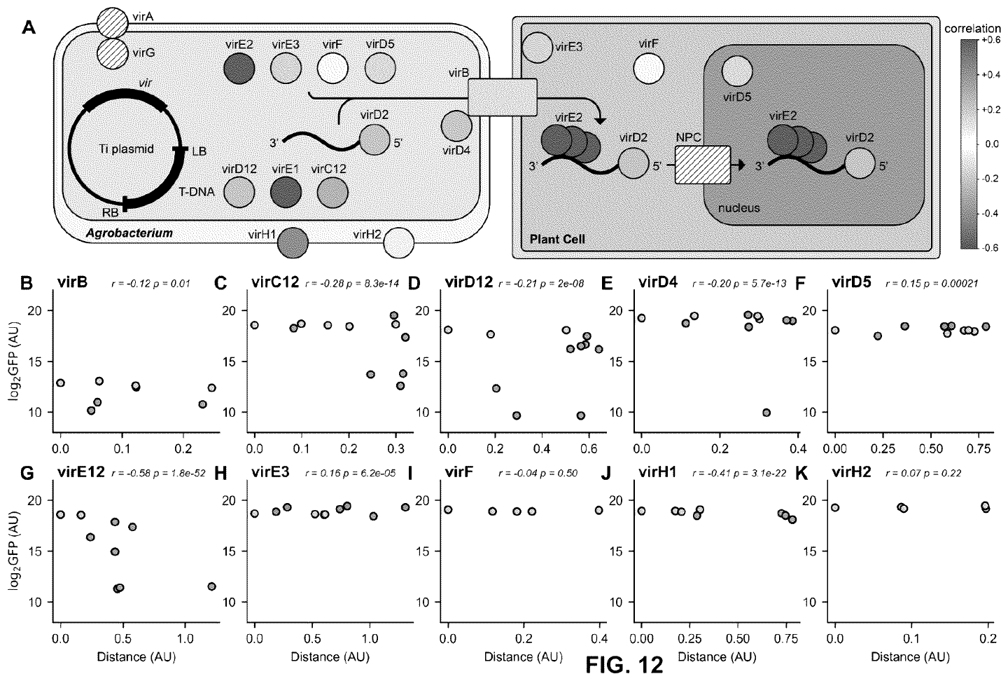

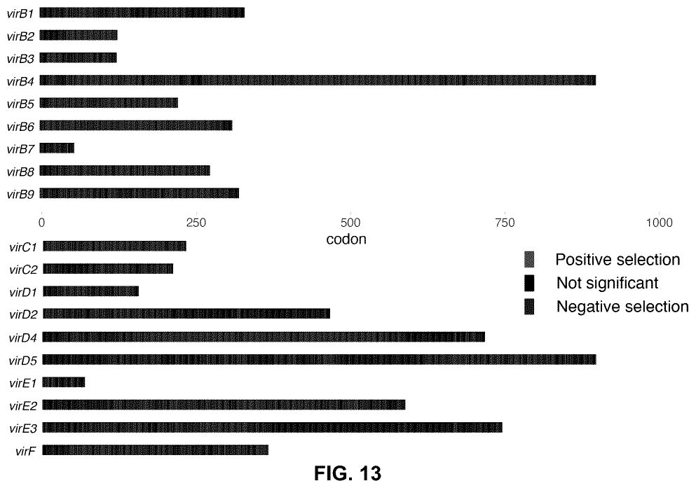

To more specifically test whether phylogenetic distance from the wild-type allele impacts the ability for a vir gene to function in a non-native system, we attempted to correlate phylogenetic distance from the C58 to the ability of a homolog to complement its deletion mutant in tobacco. However, with the exception of virE12 there were no significant correlations between phylogenetic distance and ability to complement (FIG. 12). Even distantly related alleles were able to complement many of the vir gene mutants to the level of the wild-type allele. A possible explanation of this may be that many of the vir genes appear to be under purifying selection across much of their coding sequence when analyzed by dN/dS (FIG. 13). This selective pressure may keep critical residues needed for protein-protein interactions intact across evolutionary time, but further analysis will be required to identify whether such residues exist.

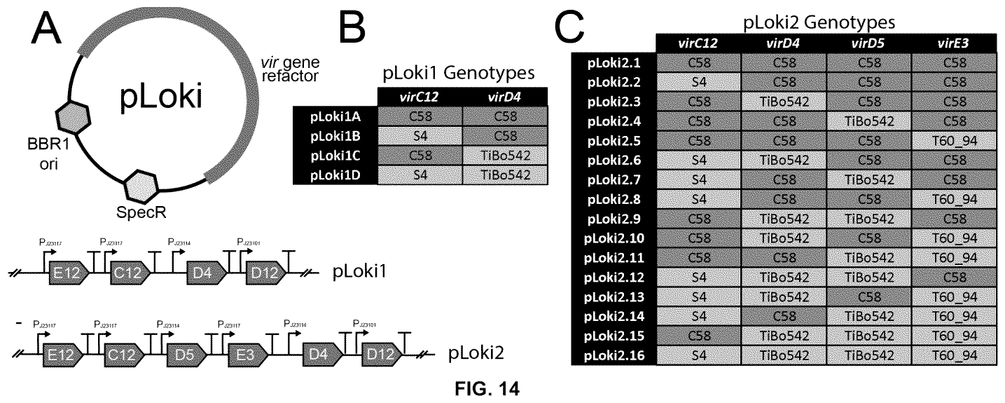

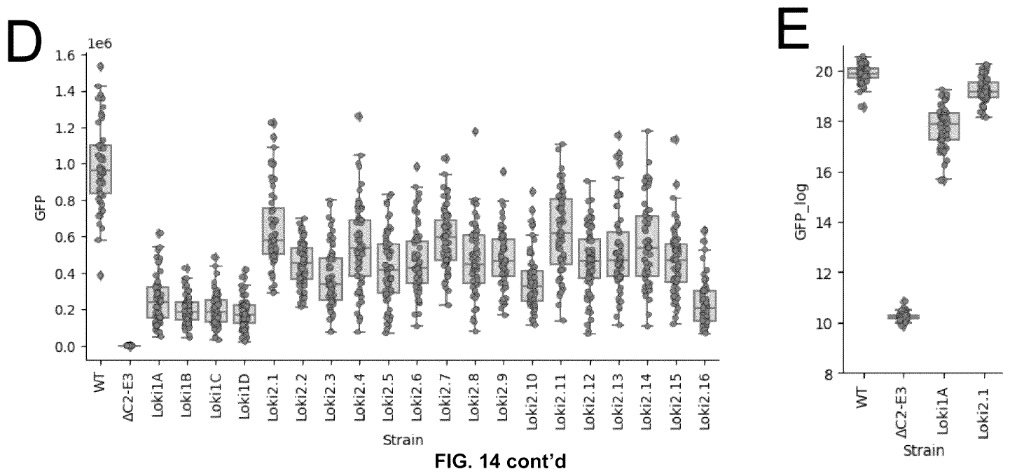

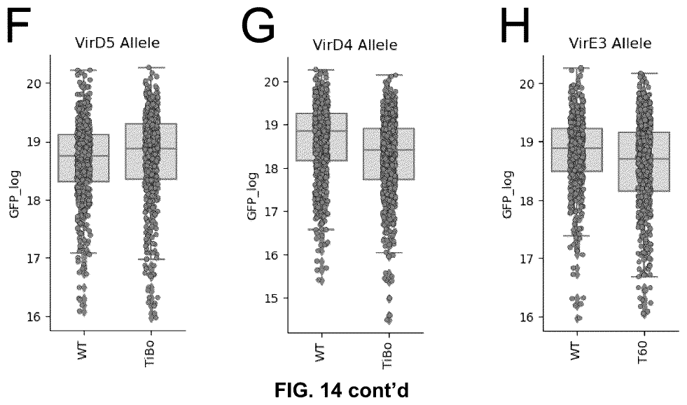

Given that we identified multiple homologs across 4 vir gene clusters that could improve transformation, we then asked if these homologs could be combined to further improve transformation. To this end we generated a suite of plasmids, called pLoki, that contained either the critical genes virC12, virD12, virD4, and virE12 (pLoki1) or these critical genes in addition to virD5 and virE3 (pLoki2) (FIG. 14, Panels A-C). We constructed a total of 20 variants of both pLoki1 and pLoki2 that explored all possible combinations of both wild-type alleles (C58) and the alleles of virC12, virD4, virD5, and virE3 that performed best from our initial screen. These vectors were used to complement a deletion that spanned virC2 to virE3 in A. fabrum GV3101 (FIG. 14, Panel D). The pLoki1 variant containing all C58 alleles restored ˜25% of wild-type transformation in a transient tobacco expression assay, whereas the pLoki2 variant containing all C58 alleles restored ˜65% (FIG. 14, Panel E). Across all pLoki variants that were generated, none that contained a non-native allele outperformed the pLoki plasmids that only contained wild-type genes (FIG. 14, Panel D). Looking across pLoki variants, we observed that when all data was consolidated that vectors containing virD5 derived from pTiBo542 were statistically superior to those harboring the wild-type (FIG. 14, Panel F), though the improvement was relatively minimal. Strains that contained virD4 from pTiBo542 or virE3 from pTiT60_94, however, were both statistically worse than strains with the corresponding wild-type allele (FIG. 14, Panels G-H). Though we were unable to improve transformation through the additive combination of promising vir homologs, harnessing allelic diversity likely represents a promising avenue for improving AMT. Future work that focuses on the specific mechanisms of our observed homolog improvements will be critical in taking full advantage of the natural diversity of vir genes.

Engineering a synthetic pTi. To exert predictable phenotypic control over AMT the genotypic and regulatory makeup of a synthetic pTi must be composed of a defined set of genes controlled by promoters that are orthogonal to regulatory influence exerted by the plant environment. Based on our quantitative assessment of vir gene importance for tobacco transformation (FIG. 2) and using optimal promoters previously identified (FIG. 1), we first sought to identify the minimal set of genetic elements capable of plant transformation, from which other designs could be based upon. We proceed to build pDimples0, which contains a minimal set of essential vir genes (i.e., virB1-11, virD12, and virD4) based on both our findings and previous work, which expressed the vir genes as a single operon controlled by P_{LacO}, and the other genes controlled by optimally determined constitutive promoters (FIG. 4, Panel A). This vector was then introduced into A. fabrum C58C1, a strain of A. fabrum which has been cured of its pTi, also harboring a binary vector expressing GFP on the T-DNA. When this strain was introduced into tobacco leaves, however, there was no measurable increase in GFP signal when compared to leaves infiltrated with A. fabrum C58C1 carrying only the binary vector (FIG. 4, Panel B). From this design we then generated two additional vectors (pDimples0.5), which added either critical genes virC12 or virE12 upstream up the virB cluster. While pDimples0.5-virC12 was unable to achieve any measurable tobacco transformation, pDimples0.5-virE12 generated GFP statistically above control (FIG. 4, Panel B). This was corroborated by experiments which expressed a nuclear localized mScarlet from the T-DNA, where C58C1 containing the minimal pDimples0.5-virE12 were able to form bright nuclear fluorescence in tobacco leaves indicating successful T-DNA transfer into plant nuclei (FIG. 4, Panel C). This experimentally demonstrates a minimal set of genes required for AMT of tobacco leaves, and a starting point for rational engineering of synthetic pTi vectors.

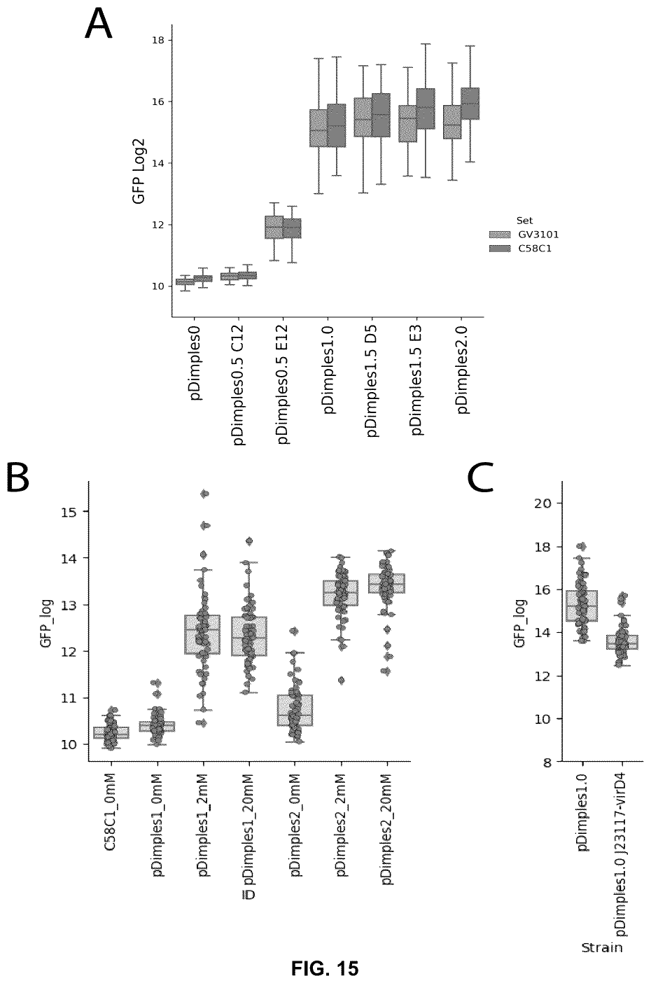

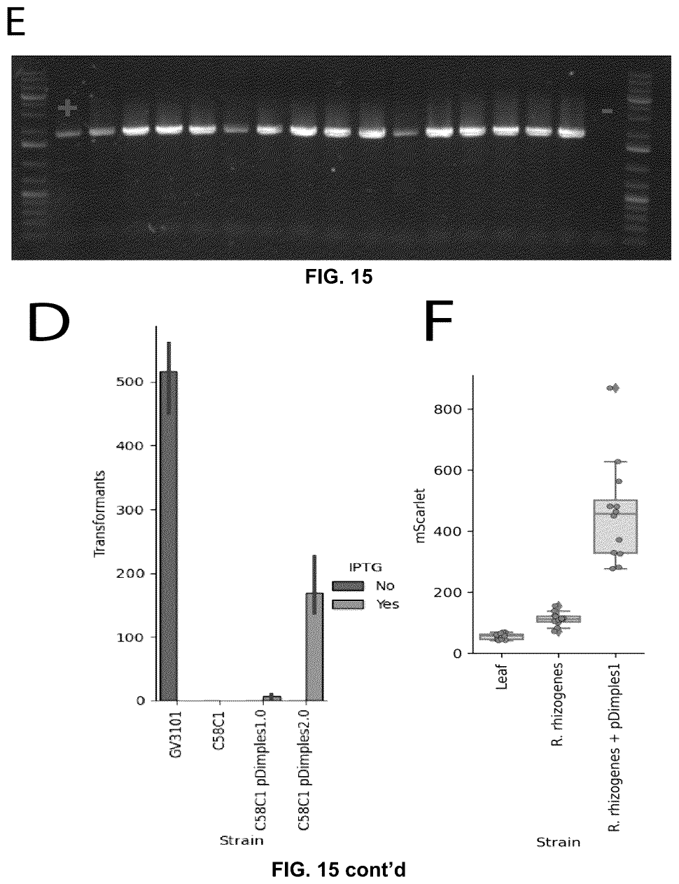

To iterate and further optimize this design, we then added both virC12 and virE12 upstream of the virB cluster (pDimples1.0) which dramatically improved transformation efficiency to 6.3% of wild type (FIG. 4, Panels B-C). We then sought to evaluate whether the addition of either effector vir genes virE3 or virD5, both of which significantly decreased transformation when deleted in A. fabrum GV3101, could improve transformation compared to pDimples1.0. Either gene was cloned in between virC12 and virB clusters to generate pDimples1.5. While pDimples1.5-virE3 statistically improved transformation over pDimples1.0 to 8.3% of wild-type, though pDimples1.5-virD5 did not statistically improve over pDimples1.0 (FIG. 4, Panel B). When both virE3 and virD5 were added to create pDimples 2.0, transformation efficiency reached 9.1% of wild-type. While this was significantly improved from pDimples1.0, it was not significantly better than the addition of virE3 alone (FIG. 4, Panel B). When these vectors were introduced into GV3101 with a deletion from virA-virE3 constituting the majority of the vir genes and their essential positive regulators, there was no significant difference in the transformation ability of each vector when compared to the results from C58C1. This suggests that—at least in the context of transient expression within tobacco—other genes on pTi may not play a significant role in the transformation process (FIG. 15, Panel A).

Attempts to optimize the expression of virB via complementation assays showed that P_{LacO} was an optimal choice to control the expression of the T4SS. The choice of the inducible P_{LacO} also allowed us to control the magnitude of transformation by the amount of IPTG that was co-infiltrated (FIG. 15, Panel B). Thus our engineering efforts allowed for controlled transformation, orthogonal to the plant environment. As the ability of pDimples vectors to restore transformation was significantly less than that of the pLoki vectors (˜10% versus 75% restoration of wild-type A. farbrum GV3101 transformation), we concluded that suboptimal expression of the T4SS was likely a limiting factor. A possible bottleneck could be the availability of virD4 which acts as a bridge between VirD2-conjugated T-DNA and the rest of the T4SS. We sought to see if virD4 expression limited transformation by replacing the very weak P_{J23114} promoter with the slightly stronger P_{J23117} promoter, however this resulted in a significant decrease in transformation, indicating the bottleneck exists elsewhere (FIG. 15, Panel C). While no pDimples vector was able to restore wild-type level transformation to A. fabrum C58C1, pDimples1 and pDimples2 outperformed any attempt to complement a virB1-11 deletion. These results are consistent with our hypothesis that a specific ratio between the T4SS genes and other vir genes needs to be maintained for optimal transformation, and exploring this relationship further will likely be key in debottlenecking further engineering efforts.

Since Agrobacterium is also a critical tool for the transformation of many fungi 34, we evaluated the ability of the pDimples vectors to transform the oleaginous yeast Rhodospordium toruloides. Unlike tobacco, a small number of transformants were observed with pDimples0.5-virC12 added, while no transformants were observed with pDimples0.5-virE12 (FIG. 4, Panel D). This is consistent with reports that virE12 is not as important for fungal transformation as it is for plant transformation 35. While only 5% of wild type transformation efficiency was achieved with pDimples1.0, but the addition of virD5 dramatically increased transformation efficiency to 40% of wild-type (FIG. 4, Panel D). This is intriguing because while VirD5 was previously shown to localize to the nucleus of fungi 29, it was thought to be completely dispensable for fungal transformation 35. Coupled with our findings that there is little diversity selection within the coding region of virD5, it is likely that this gene has a far more fundamental role in AMT than simply as a determinant of host range. The addition of virE3 by itself did not improve transformation, nor did it improve transformation efficiency when added in combination with virD5. As with tobacco experiments, transformation was dependent on the presence of IPTG to induce virB1-11 expression (FIG. 15, Panel D), with R. toruloides transformants being confirmed by colony PCR (FIG. 15, Panel E).

To test whether a synthetic pTi is sufficient to impart AMT outside of its native context, we sought to test our engineered designs in a bacterium beyond A. farbrum. To this end we introduced pDimples1.0 into Rhizobium rhizogenes D108/85, which was isolated without a native pRi or pTi plasmid that last shared a common ancestor with A. fabrum ˜200 million years ago 33. When R. rhizogenes was infiltrated into tobacco leaves carrying a binary vector expressing nuclear-localized mScarlet, no red nuclei were observed, yet with the addition of pDimples1.0 clear red nuclei were observed that produced significantly more fluorescent signal than the parent strain (FIG. 4, Panel E; FIG. 15, Panel F). Together, our results demonstrate that the design and construction of synthetic pTi can be used to: 1) identify the core set of genes that are necessary and sufficient for AMT, 2) the contributory role of accessory vir genes, 3) divorce AMT from its native regulation, and 4) transfer this complex trait into other bacteria.

### CONCLUSION

By leverate a comprehensive and quantitative understanding of each vir gene cluster, we have built synthetic pTi plasmids that define the minimal transferable system required for AMT of both plants and fungi. Optimization of these systems will allow us to better understand host-specificity between natural strains of agrobacteria, and engineer laboratory strains with superior transformation properties. Furthermore, our analysis of how allelic variation within vir genes impacts transformation suggests there are likely untapped genetic resources to improve AMT. Overall, this work will also serve to guide related research studying host-microbe interactions, specifically those of plant-associated bacteria. For example, recent research that developed minimized versions of the nitrogen fixing pSymA in the root nodule-associated legume symbiont Sinorhizobium meliloti could be furthered by evaluating the impact of gene expression on individual genes 12.

Assessing bacterial synthetic biology parts both in vitro and in multiple plant species revealed that while constitutive synthetic promoters will likely perform similarly in different environments, the performance of inducible systems may be highly variable. Further characterization of synthetic regulatory elements in situ will enable more precise engineering. However, by using these tools to replace the master regulatory VirA/G system with synthetic regulation, we not only gain precise control of individual gene expression, but also insulate the bacteria from attempts by the host to interfere with gene expression, which has been previously observed 36,37. Separating AMT induction from its native inducing conditions (i.e., low pH, sugar, and phenolic compounds) may also provide unique opportunities in improving fungal transformations, which currently require long induction times in these conditions and may not be optimal for the growth of certain fungi 35,38.

Our ability to transfer the transformation phenotype via pDimples into R. rhizogenes opens the door to another promising avenue of AMT engineering: transferring the complex vir machinery to other bacteria. As A. fabrum is known to elicit strong plant immune responses that impede transformation, multiple efforts have been made recently to circumvent this either through mutation of known immunogenic loci 39 or the addition of immune suppressing systems 40. Our work lays the foundation to developing synthetic pTi that function in bacteria that elicit minimal immune responses, potentially enabling the transformation of plant species and cultivars that have traditionally been recalcitrant to genetic modification. Our inability to efficiently transform new organisms represents the biggest bottleneck to dramatically expanding the scope and range of species that can be utilized for synthetic biology. Given the wide diversity of eukaryotes that can be transformed by Agrobacterium, future synthetic pTi may be optimized to target currently untransformable organisms and enable entirely new areas of biotechnology.

### Materials and Methods

Media, chemicals, and culture conditions. Routine bacterial cultures were grown in Luria-Bertani (LB) Miller medium (BD Biosciences, USA). E. coli was grown at 37° C., while A. fabrum was grown at 30° C. unless otherwise noted. Cultures were supplemented with kanamycin (50 mg/L, Sigma Aldrich, USA), gentamicin (30 mg/L, Fisher Scientific, USA), or spectinomycin (100 mg/L, Sigma Aldrich, USA), when indicated. All other compounds unless otherwise specified were purchased through Sigma Aldrich. Bacterial kinetic growth curves were performed as described previously 26.

Strains and plasmids. All bacterial strains and plasmids used in this work are listed in Supplemental Table 1 and 2. All strains and plasmids created in this work are viewable through the public instance of the JBEI registry. (webpage for: registry.jbei.org/folders/2424). All plasmids generated in this paper were designed using Device Editor and Vector Editor software, while all primers used for the construction of plasmids were designed using j5 software 41-43. Synthetic DNA was synthesized from Twist Biosciences. Plasmids were assembled via Gibson Assembly using standard protocols 44, Golden Gate Assembly using standard protocols 45, or restriction digest followed by ligation with T4 ligase as previously described 46. Plasmids were routinely isolated using the Qiaprep Spin Miniprep kit (Qiagen, USA), and all primers were purchased from Integrated DNA Technologies (IDT, Coralville, IA). Plasmid sequences were verified using whole plasmid sequencing (Primordium Labs, Monrovia, CA). Agrobacterium was routinely transformed via electroporation as described previously 47.

Construction of deletion mutants. Deletion mutants in A. fabrum GV3101 were constructed by homologous recombination and sacB counterselection using the allelic exchange as described previously 48. Briefly, homology fragments of 1 kbp up- and downstream of the target gene, including the start and stop codons respectively, were cloned into pMQ30K-a kanamycin resistance-bearing derivative of pMQ30 49. Plasmids were then transformed via electroporation into E. coli S17 and then mated into A. fabrum via conjugation. Transconjugants were selected for on LB Agar plates supplemented with kanamycin 50 mg/mL, and rifampicin 100 mg/mL. Transconjugants were then grown overnight on LB media also supplemented with 50 mg/mL kanamycin, and 100 mg/mL rifampicin, and then plated on LB Agar with no NaCl supplemented with 10% w/v sucrose. Putative deletions were restreaked on LB Agar with no NaCl supplemented with 10% w/v sucrose, and then were screened via PCR with primers flanking the target gene to confirm gene deletion.

Synthetic part characterization. Characterization of pGinger vectors harbored by A. fabrum in vitro was performed as previously described for other bacteria 26. Briefly, A. fabrum C58C1 with different pGinger vectors were grown overnight in 10 mL of LB supplemented with kanamycin overnight at 30° C. with 250 rpm shaking and then diluted 1:100 into 500 μL of fresh LB media with kanamycin in a deep-well 96-well plate (Corning) For inducible promoters, chemical inducers were added in two-fold dilutions before incubation. Cells were then grown at 30° C. for 24-hours while shaking at 250 rpm, and then 100 μL was measured for absorbance at OD₆₀₀ as well as for RFP fluorescence using an excitation wavelength of 590 nm and an emission wavelength of 635 nm with a gain setting of 75 on a BioTek Synergy H1 microplate reader (Agilent).

To evaluate the performance of synthetic promoters in planta, strains were grown in 5 mL LB media with kanamycin at 30° C. with 250 rpm shaking overnight, and then diluted 1:5 with fresh media then grown for an additional 3 hours at 30° C. with 250 rpm shaking. Cultures were then adjusted to an absorbance at OD₆₀₀ of 1.0 in agroinfiltration buffer (10 mM MgCl₂, 10 mM MES, 200 μM acetosyringone, pH 5.6), and infiltrated into either N. benthamiana or A. thaliana leaf tissue. When appropriate chemical inducers were added to the agroinfiltration media immediately before leaf infiltration. Either one, or three days post-infiltration 6 mm leaf disks were excised from each agroinfiltrated leaf using a hole puncher and placed atop 300 μL of water in a black, clear-bottom, 96-well microtiter plate (Corning). GFP fluorescence of each leaf disk was then measured using a BioTek Synergy H1 microplate reader (Agilent) with an excitation wavelength of 488 nm and measurement wavelength of 520 nm.

Plant Growth Conditions. A. thaliana were germinated and grown in Sunshine Mix #1 soil (Sungro) in a Percival growth chamber at 22° C. and 60% humidity using a 8/16 hour light/dark cycle with a daytime PPFD of ˜200 μmol/m²s. N. benthamiana plants were grown according to a previously described standardized lab protocol 27. All tobacco growth was conducted in an indoor growth room at 25° C. and 60% humidity using a 16/8 hour light/dark cycle with a daytime PPFD of ˜120 μmol/m²s. Plants were maintained in Sunshine Mix #4 soil (Sungro) supplemented with Osmocote 14-14-14 fertilizer (ICL) at 5 mL/L and agroinfiltrated 29 days after seed sowing.

Tobacco Infiltration and Leaf Punch Assay. A. fabrum strains were grown in LB liquid media containing necessary antibiotics (50 μg/mL rifampicin, 30 μg/mL gentamicin, 50 μg/mL kanamycin, and 100 μg/mL spectinomycin for most strains) to an OD600 between 0.6 and 1.0 before pelleting. Cells were then prepared for infiltration by resuspension in agroinfiltration buffer (10 mM MgCl₂, 10 mM MES, 200 μM acetosyringone, pH 5.6) to a final OD600 of 1.0 and were allowed to induce for 2 hours in infiltration buffer at room temperature. When appropriate, chemical inducers (i.e. IPTG) were added during the 2 hour induction period. Each strain was then infiltrated into the fourth and fifth leaf (counting down from the top) of eight biological replicate tobacco plants. GFP transgene expression in agroinfiltrated leaves was then assessed by a leaf disk fluorescence assay three days post-infiltration. Four 6 mm leaf disks were excised from each agroinfiltrated leaf using a hole puncher and placed atop 300 μL of water in a black, clear-bottom, 96-well microtiter plate (Corning). GFP fluorescence of each leaf disk was then measured using a BioTek Synergy H1 microplate reader (Agilent) with an excitation wavelength of 488 nm and measurement wavelength of 520 nm.

Rhodospordium toruloides Transformation. Agrobacterium tumefaciens mediated transformation was performed on Rhodosporidium toruloides IFO0880 with a codon optimized epi-isozizaene synthase from Streptomyces coelicolor A3 (2) (JPUB_013523) 50 as previously described 51. When appropriate 2 mM IPTG was added to agrobacterium induction media. Transformants were confirmed via colony PCR specific to the integrated T-DNA.

Proteomic Analysis. Proteins from A. fabrum samples were extracted using a previously described chloroform/methanol precipitation method 52. Extracted proteins were resuspended in the 100 mM ammonium bicarbonate buffer supplemented with 20% methanol, and protein concentration was determined by the DC assay (BioRad). Protein reduction was accomplished using 5 mM tris 2-(carboxyethyl) phosphine (TCEP) for 30 min at room temperature, and alkylation was performed with 10 mM iodoacetamide (IAM; final concentration) for 30 min at room temperature in the dark. Overnight digestion with trypsin was accomplished with a 1:50 trypsin: total protein ratio. The resulting peptide samples were analyzed on an Agilent 1290 UHPLC system coupled to a Thermo scientific Obitrap Exploris 480 mass spectrometer for discovery proteomics 53. Briefly, 20 μg of tryptic peptides were loaded onto an Ascentis® (Sigma-Aldrich) ES-C18 column (2.1 mm×100 mm, 2.7 μm particle size, operated at 60° C.) and were eluted from the column by using a 10 minute gradient from 98% buffer A (0.1% FA in H2O) and 2% buffer B (0.1% FA in acetonitrile) to 65% buffer A and 35% buffer B. The eluting peptides were introduced to the mass spectrometer operating in positive-ion mode. Full MS survey scans were acquired in the range of 300-1200 m/z at 60,000 resolution. The automatic gain control (AGC) target was set at 3e6 and the maximum injection time was set to 60 ms. Top 10 multiply charged precursor ions (2-5) were isolated for higher-energy collisional dissociation (HCD) MS/MS using a 1.6 m/z isolation window and were accumulated until they either reached an AGC target value of 1e5 or a maximum injection time of 50 ms. MS/MS data were generated with a normalized collision energy (NCE) of 30, at a resolution of 15,000. Upon fragmentation precursor ions were dynamically excluded for 10 s after the first fragmentation event. The acquired LCMS raw data were converted to mgf files and searched against the latest uniprot A. tumefaciens protein database with Mascot search engine version 2.3.02 (Matrix Science). The resulting search results were filtered and analyzed by Scaffold v 5.0 (Proteome Software Inc.). The normalized spectra count of identified proteins were exported for relative quantitative analysis.

Bioinformatic Analyses. Sequences of individual vir genes from genomes of all sequenced agrobacteria were identified and extracted as previously described 54. MACSE v. 2.07 with the parameter “-prog alignSequences” was used to generate codon alignments for each vir gene dataset 55. The HYPHY v2.2 program “cln” was used to remove identical sequences and stop codons from each alignment 56. IQ-TREE v. 1.6.12 with the default parameters was used to generate a phylogeny for each dataset 57. The HYPHY program FUBAR with the codon alignment, phylogeny, and a probability threshold of 0.9 was used to calculate per-site dN/ds and detect signals of positive or purifying selection.

Statistical analyses and data presentation. All numerical data were analyzed using custom Python scripts. All graphs were visualized using either Seaborn or Matplotlib 58,59. Calculation of 95% confidence intervals, standard deviations, and T-test statistics were conducted via the Scipy library 60. Bonferroni corrections were calculated using the MNE python library 61. Alleles of homologus vir genes were aligned using MAFFT v. 7.508 62 and converted into phylogenetic trees using FastTree v. 2.1.11 63. Phylogenetic distance was calculated using dendropy v. 4.6.1 64.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A nucleic acid encoding refactored minimized set of Agrobacterium virulence genes.

2. The nucleuc acid of claim 1, wherein the refactored minimized set of Agrobacterium virulence genes comprises the following genes: virB1, virB2, virB3, virB4, virB5, virB6, virB7, virB8, virB9, virB10, virB11, virD4, and virD12, and virE12 or Agrobacterium rhizogenes GALLS gene.

3. The nucleuc acid of claim 2, wherein the refactored minimized set of Agrobacterium virulence genes at least excludes: virA, virG, virB1, or virE12 genes.

4. The nucleuc acid of claim 3, wherein the refactored minimized set of Agrobacterium virulence genes at least excludes: virA, virG, and virB1 genes.

5. The nucleuc acid of claim 2, wherein the refactored minimized set of Agrobacterium virulence genes comprises virC12, virD5, or virE3.

6. The nucleuc acid of claim 5, wherein the refactored minimized set of Agrobacterium virulence genes comprises virC12.

7. The nucleuc acid of claim 5, wherein the refactored minimized set of Agrobacterium virulence genes comprises virD5 or virE3.

8. The nucleuc acid of claim 7, wherein the refactored minimized set of Agrobacterium virulence genes comprises virD5 and virE3.

9. A vector comprising the nucleic acid of claim 1, wherein the genes are operatively linked to one or more promoters.

10. The vector of claim 9, wherein the vector is capable of stably integrating into a chromosome of a host cell or stably residing in a host cell.

11. A host cell comprising the vector of claim 10, wherein the vector is stably integrated into a chromosome of the host cell or is stably residing in the host cell.

12. A method for introducing a nucleic acid of interest into a eukaryotic cell, the method comprises: (a) providing (i) a first nucleic acid encoding a refactored minimized set of Agrobacterium virulence genes comprises the following genes: virB1, virB2, virB3, virB4, virB5, virB6, virB7, virB8, virB9, virB10, virB11, virD4, and virD12, and virE12 or Agrobacterium rhizogenes GALLS gene, which are operatively linked to one or more promoters; and (ii) a second nucleic acid comprising a nucleic acid of interest flanked by a left border and a right border; (b) introducing the first nucleic acid and the second nucleic acid into a target host cell; and, (c) the nucleic acid of interest is stably integrated into a genome of the target host cell.

13. The method of claim 12, wherein the first nucleic acid and the second nucleic acid reside on a single nucleic acid molecule capable of stably residing in the host cell.

14. The method of claim 13, wherein the single nucleic acid molecule is a minimal refactored pTi plasmid.

15. The method of claim 13, wherein the single nucleic acid molecule is stably integrated in a chromosome of the host cell.

16. The method of claim 12, wherein nucleic acid of interest encodes one or more genes of interest (GOI) each operatively linked to a promoter capable of expression in the host cell.

17. A method for constructing a refactored minimized set of Agrobacterium virulence genes compring: ligating or synthesizing a nucleic acid encoding a refactored minimized set of Agrobacterium virulence genes comprising the following genes: virB1, virB2, virB3, virB4, virB5, virB6, virB7, virB8, virB9, virB10, virB11, virD4, and virD12, and virE12 or Agrobacterium rhizogenes GALLS gene.
